(19) United States
(12) Patent Application Publication
(10) Pub. No.: US 2014/0113376 A1
(43) Pub. Date: Apr. 24, 2014

(54) COMPOSITIONS AND METHODS FOR DOWNREGULATING PROKARYOTIC GENES

(71) Applicants: Rotem Sorek, Rehovot (IL); Gil Amitai, Rehovot (IL)

(72) Inventors: Rotem Sorek, Rehovot (IL); Gil Amitai, Rehovot (IL)

(21) Appl. No.: 14/123,267

(22) PCT Filed: May 31, 2012
(86) PCT No.: PCT/IL12/50194

§ 371 Date: Dec. 2, 2013

Related U.S. Application Data
(60) Provisional application No. 61/491,943, filed on Jun. 1, 2011.

(51) Int. Cl. C07K 14/195

(52) U.S. Cl. CPC C07K 14/195
USPC 435/471; 536/23.7

(57) ABSTRACT

An isolated polynucleotide is disclosed. The polynucleotide comprises a clustered, regularly interspaced short palindromic repeat (CRISPR) array nucleic acid sequence wherein at least one spacer of the CRISPR is sufficiently complementary to a portion of at least one prokaryotic gene so as to down-regulate expression of the prokaryotic gene and further comprises a nucleic acid sequence encoding at least one CRISPR associated (CAS) polypeptide of a repeat associated mysterious protein (RAMP) family. Uses of the polynucleotides and pharmaceutical compositions comprising the polynucleotides are also disclosed.

### FIELD AND BACKGROUND OF THE INVENTION

The present invention, in some embodiments thereof, relates to methods of downregulating prokaryotic genes and, more particularly, but not exclusively, to methods of downregulating bacterial genes.

One of the most successful ways for understanding the function of a gene within an organism is to silence its expression. This is usually done by modifying, interrupting or deleting the DNA of the gene, and is referred to as gene ‘knockout’ [Austin et al., 2004, Nature Genetics 36:921-4]. Gene knockouts are mostly carried out through homologous recombination, and this method was used in multiple studies and in multiple organisms from bacteria to mammals.

Despite the fact that gene targeting by knockout is a powerful tool, it is a complex, labor intensive and time consuming procedure. It is therefore hard to scale up cost-effectively, and most studies are generally limited to a knockout of a single gene rather than a complete pathway. Moreover, knockout is mostly limited to organisms in which homologous recombination is relatively efficient, such as mouse [Austin et al., 2004, Nature Genetics 36:921-4] and yeast [Deutscher et al., 2006, Nature Genetics 38:993-8].

A breakthrough in the search for efficient alternative to gene knockouts in eukaryotes was achieved when it was realized that RNA-interference (RNAi) could be used to silence the expression of specific genes without interruption of their DNA. RNAi is a conserved biological mechanism first discovered in the nematode Caenorhabditis elegans, where it was demonstrated that injection of long dsRNA into this nematode led to sequence-specific degradation of the corresponding mRNAs. This silencing response has been subsequently found in other eukaryotes including fungi, plants and mammals [Fire et al., 1998, Nature 391(6669):806-11; Hannon, 2002, Nature 418(6894):244-51].

While the role of RNAi is, at least in part, to protect against viral infections and mobile element infestations, it was also shown that artificial transfection of short dsRNA duplexes, which target specific endogenous mRNAs, into mammalian cells can trigger gene specific silencing [Elbashir et al., 2001, Nature. 2001 May 24; 411(6836):494-8]. These short dsRNAs (called siRNAs) are converted into single strands by the RNA-induced silencing protein complex (RISC), and the RISC-siRNA complex identifies target mRNAs by base pairing, leading to their degradation by an RNA nuclease. Researchers are now using this RNAi gene silencing technology and its derivatives to understand the biological function of endogenous genes in many eukaryotic organisms, and usage of this technology has led to important scientific breakthroughs.

The enormous advantages of RNAi in genetic studies have so far not been reproduced in prokaryotes (bacteria and archaea), because the RNAi system seems to be limited to the eukaryotic lineage.

U.S. Patent Application 20040053289 teaches the use of si hybrids to down-regulate prokaryotic genes.

CRISPR is a genetic system comprised of a cluster of short repeats (24-47 bp long), interspersed by similarly sized non repetitive sequences (called spacers). Additional components of the system include CRISPR-associated (CAS) genes and a leader sequence (FIG. 1A). This system is abundant among prokaryotes, and computational analyses show that CRISPRs are found in ˜40% of bacterial and ˜90% of archaeal genomes sequenced to date [Grissa et al, 2007, BMC Bioinformatics 8: 17].

CRISPR arrays and CAS genes vary greatly among microbial species. The direct repeat sequences frequently diverge between species, and extreme sequence divergence is also observed in the CAS genes. The size of the repeat can vary between 24 and 47 bp, with spacer sizes of 26-72 bp. The number of repeats per array can vary from 2 to the current record holder, Haliangium ochraceum, which has 382 repeats in one array and, although many genomes contain a single CRISPR locus, M. jannaschii has 18 loci. Finally, although in some CRISPR systems only 6, or fewer, CAS genes have been identified, others involve more than 20. Despite this diversity, most CRISPR systems have some conserved characteristics (FIG. 1A).

It was recently demonstrated experimentally that in response to phage infection, bacteria integrate new spacers that are derived from phage genomic sequences, resulting in CRISPR-mediated phage resistance. The new repeat-spacer units were added at the leader-proximal end of the array, and had to match the phage sequence exactly (100% identity), to provide complete resistance. When such phage-derived spacers were artificially introduced into the CRISPR array of a phage-sensitive S. thermophilus strain, it became phage-resistant [Barrangou et al, 2007, Science 315(5819): 1709-12]. Indeed, spacers found in naturally occurring CRISPR arrays are frequently derived from phages and other extrachromosomal elements [Bolotin et al., 2005, Microbiology 151(Pt 8): 2551-61].

Additional background art includes Horvath et al [International Publication No. WO2008/108989, Bronus et al [Science, 321, 960 (2008)], U.S. Application No: 20100076057 and Sorek et al., [Nature Reviews Microbiology, 6, 181, 2008].

### SUMMARY OF THE INVENTION

According to an aspect of some embodiments of the present invention there is provided an isolated polynucleotide, comprising

(i) a clustered, regularly interspaced short palindromic repeat (CRISPR) array nucleic acid sequence wherein at least one spacer of the CRISPR is sufficiently complementary to a portion of at least one prokaryotic gene so as to down-regulate expression of the prokaryotic gene; and

(ii) a nucleic acid sequence encoding at least one CRISPR associated (CAS) polypeptide of a repeat associated mysterious protein (RAMP) family.

According to an aspect of some embodiments of the present invention there is provided a nucleic acid construct comprising the isolated polynucleotide of the present invention.

According to an aspect of some embodiments of the present invention there is provided a nucleic acid construct system comprising:

(i) a first nucleic acid construct comprising an isolated polynucleotide having a clustered, regularly interspaced short palindromic repeat (CRISPR) array nucleic acid sequence wherein at least one spacer of the CRISPR is sufficiently complementary to a portion of at least one prokaryotic gene so as to down-regulate expression of the prokaryotic gene; and

(ii) a second nucleic acid construct comprising an isolated polynucleotide having a nucleic acid sequence encoding at least one CRISPR associated (CAS) polypeptide of a repeat associated mysterious protein (RAMP) family.

According to an aspect of some embodiments of the present invention there is provided a method of down-regulating expression of a gene of a prokaryotic cell, the method comprising introducing into the cell a CRISPR system polynucleotide encoding a CRISPR array and at least one CRISPR associated (CAS) polypeptide of a repeat associated mysterious protein (RAMP) family, wherein a spacer of the CRISPR array is sufficiently complementary with a portion of the gene to down-regulate expression of the gene, thereby down-regulating expression of gene of a prokaryotic cell.

According to an aspect of some embodiments of the present invention there is provided a nucleic acid construct comprising two repeat sequences of a CRISPR array flanking a cloning site or making a cloning site when concatenated.

According to an aspect of some embodiments of the present invention there is provided a method of treating a bacterial infection in a subject in need thereof, the method comprising administering to the subject a therapeutically effective amount of an isolated polynucleotide, comprising a clustered, regularly interspaced short palindromic repeat (CRISPR) system nucleic acid sequence, the CRISPR system encoding a CRISPR array and at least one CRISPR associated (CAS) polypeptide of a repeat associated mysterious protein (RAMP) family wherein at least one spacer of the CRISPR array is sufficiently complementary to a portion of at least one bacterial gene so as to down-regulate expression of the bacterial gene, the bacterial gene being a vital bacterial gene or a bacterial virulence gene, thereby treating the bacterial infection.

According to an aspect of some embodiments of the present invention there is provided a method of treating an antibiotic-resistant bacterial infection in a subject in need thereof, the method comprising administering to the subject the isolated polynucleotide of the present invention, thereby treating the antibiotic-resistant bacterial infection.

According to an aspect of some embodiments of the present invention there is provided a method of annotating a prokaryotic gene, the method comprising:

(a) introducing the isolated polynucleotide of the present invention into a prokaryote under conditions that allow downregulation of the prokaryotic gene; and

(b) assaying a phenotype of the prokaryote, wherein a change in phenotype following the introducing is indicative of the prokaryotic gene being associated with the phenotype.

According to some embodiments of the invention, the at least one spacer comprises at least two spacers, each being sufficiently complementary to a portion of different prokaryotic genes so as to down-regulate expression of the different prokaryotic genes.

According to some embodiments of the invention, the at least one spacer comprises 26-72 base pairs.

According to some embodiments of the invention, the prokaryotic gene is a bacterial gene.

According to some embodiments of the invention, the bacterial gene is associated with down-regulation of biofuel production.

According to some embodiments of the invention, the bacterial gene is selected from the group consisting of acetate kinase, phosphate acetyltransferase and L-lactate dehydrogenase.

According to some embodiments of the invention, the bacterial gene is a genetic repressor CcpN.

According to some embodiments of the invention, the bacterial gene is an antibiotic resistance gene.

According to some embodiments of the invention, the antibiotic resistance gene is a methicillin resistance gene or a vancomycin resistance gene.

According to some embodiments of the invention, the bacterial gene is a bacterial virulence gene.

According to some embodiments of the invention, the bacterial gene is a ribosomal RNA gene, a ribosomal protein gene or a tRNA synthestase gene.

According to some embodiments of the invention, the bacterial gene is selected from the group consisting of dnaB, fabI, folA, gyrB, murA, pytH, metG, and tufA(B).

According to some embodiments of the invention, the prokaryotic gene is an archaeal gene.

According to some embodiments of the invention, the isolated polynucleotide further comprises a nucleic acid sequence encoding a CRISPR leader sequence.

According to some embodiments of the invention, the mesophilic organism is Neisseria sicca ATCC29256.

According to some embodiments of the invention, the nucleic acid construct comprises a nucleic acid sequence encoding at least one polypeptide having at sequence selected from the group consisting of SEQ ID NOs: 1354-1360.

According to some embodiments of the invention, the nucleic acid construct encodes each of the polypeptides as set forth in SEQ ID NOs: 1354-1360.

According to some embodiments of the invention, the at least one CAS polypeptide comprises an amino acid sequence selected from the group consisting of SEQ ID NOs: 1354-1360.

According to some embodiments of the invention, the first nucleic acid construct comprises a leader sequence upstream to the at least one spacer as set forth in SEQ ID NO: 1374.

According to some embodiments of the invention, a repeat sequence of the CRISPR array is as set forth in SEQ ID NO: 1369, 1373, 1374 or 1375.

According to some embodiments of the invention, the nucleic acid construct further comprises a leader sequence of the CRISPR array.

According to some embodiments of the invention, CRISPR array is of a RAMP module.

According to some embodiments of the invention, the nucleic acid construct further comprises at least one spacer sequence of the CRISPR array.

According to some embodiments of the invention, the at least one CRISPR associated (CAS) polypeptide of a RAMP family is of a mesophilic organism.

According to some embodiments of the invention, the at least one CRISPR associated (CAS) polypeptide of a RAMP family is of a thermophilic organism.

According to some embodiments of the invention, the nucleic acid sequence encoding at least one CRISPR associated (CAS) polypeptide of a RAMP family comprises a sequence encoding a RAMP module.

According to some embodiments of the invention, the isolated polynucleotide is non-naturally occurring.

According to some embodiments of the invention, the nucleic acid construct comprises a nucleic acid sequence selected from the group consisting of SEQ ID NOs: 1, 6 and 11.

According to some embodiments of the invention, the nucleic acid construct comprises a cis regulatory element.

According to some embodiments of the invention, the cis regulatory element is a promoter.

According to some embodiments of the invention, the promoter is an inducible promoter.

According to some embodiments of the invention, the gene is not introduced into the cell by a bacteriophage.

According to some embodiments of the invention, the gene is integrated into a chromosome of the cell.

According to some embodiments of the invention, the gene is endogenous to the prokaryotic cell.

According to some embodiments of the invention, the gene is epichromosomal.

According to some embodiments of the invention, the method further comprises introducing into the cell a naïve CRISPR array system.

According to some embodiments of the invention, the bacterial infection is induced by methicillin resistant Staphylococcus aureus or vancomycin resistant Staphylococcus aureus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a typical structure of a CRISPR locus (system).

FIG. 1B is a model illustrating how CRISPR acquires phage-derived spacers which provide immunity (adapted from Sorek et al. Nature Reviews Microbiology, 6, 181, 2007). Following an attack by phage, phage nucleic acids proliferate in the cell and new particles are produced leading to death of the majority of sensitive bacteria. A small number of bacteria acquire phage derived spacers (blue spacer, marked by asterix) leading to survival, presumably via CRISPR-mediated degradation of phage mRNA or DNA.

FIG. 2 is a model teaching an exemplary method of silencing of self genes with an engineered RAMP (repeat associated mysterious protein) module. The RAMP module (containing the CRISPR array as well as Cas proteins) is cloned into a plasmid. Fragments from a chromosome-encoded gene (green) that is expressed to RNA the cell (green waves) are engineered into the CRISPR array as new spacers. When the engineered RAMP module is inserted inside the prokaryotic cell, the system silences the expression of the RNA of the self gene (silenced RNA is depicted in the figure as dashed green waves). The plasmid carrying the CRISPR can contain an inducible promoter, that turns the expression of the system on only in a certain condition. Thus, a conditional silencing of self genes could be achieved. The cas genes and the CRISPR array could also be cloned on two different plasmids.

FIG. 3 is a schematic drawing illustrating the organization of the Myxococcus xanthus DK 1622 Genbank AC_{—}008095 RAMP module.

FIG. 4 is a schematic drawing illustrating the RAMP module of the Myxococcus xanthus DK 1622 cloned into the pCDFDuet-1 plasmid under the control of an inducible promoter.

FIG. 5 is a schematic drawing illustrating the genomic vicinity of the RAMP module of the Myxococcus xanthus DK 1622. The illustration shows that another CRISPR system, of the Tneap subtype, is located at a nearby region of the genome.

FIG. 6 is a schematic drawing illustrating the organization of the Myxococcus xanthus DK 1622 Genbank AC_{—}008095 Tneap CRISPR system.

FIG. 7 is a polynucleotide sequence of a CRISPR construct for silencing of GFP expression in E. coli (SEQ ID NO: 1340). The sequences in red are sequences of a spacers which target the antisense strand of GFP. The highlighted yellow region shows the repeat sequence of this construct.

FIG. 8 is a polynucleotide sequence of a CRISPR construct for silencing of malF expression in E. coli (SEQ ID NO: 1341). The sequences in red are sequences of a spacers which target the antisense strand of malF. The highlighted yellow region shows the repeat sequence of this construct.

FIG. 9 is a polynucleotide sequence of a CRISPR construct for silencing of RFP expression in E. coli (SEQ ID NO: 1342). The sequences in red are sequences of a spacers which target the antisense strand of RFP. The highlighted yellow region shows the repeat sequence of this construct.

FIG. 10 is a polynucleotide sequence of a CRISPR construct for silencing of GFP and malF expression, together, in E. coli (SEQ ID NO: 1343). The sequences in red are sequence of spacers which target the antisense strand of GFP. The sequences in blue are sequence of spacers which target the sense strand of malF. The highlighted yellow region shows the repeat sequence of this construct.

FIG. 11 is a polynucleotide sequence of a control CRISPR construct which does not target any gene of interest (SEQ ID NO: 1344). The highlighted yellow region shows the repeat sequence of this construct.

FIG. 12 is a polynucleotide sequence of GFP showing the positions of the sequences targeted by an exemplary CRISPR construct in red (SEQ ID NO: 1345).

FIG. 13 is a polynucleotide sequence of malF showing the positions of the sequences targeted by an exemplary CRISPR construct in red (SEQ ID NO: 1346).

FIGS. 14A-B is a schematic representation illustrating the organization of the Neisseria sicca ATCC29256 CRISPR-RAMP module (Genbank accession No. NZ_ACK002000045). FIG. 14A—the organization of the RAMP module on the bacteria genome; FIG. 14B—the RAMP module was placed on a 3-plasmid system. A fourth plasmid (RFP/GFP) was designed as a reporter plasmid.

FIGS. 15A-B illustrates the activity of heterologous RAMP systems within E. coli. (FIG. 15A) The N. sicca RAMP module was cloned into E. coli BL21 (DE3) on a compatible plasmid system (pET-Duet) divided into 3 operons. All operons were inducible with IPTG. (FIG. 15B) Gene and crRNA expression in the tested system was induced with 0.1 mM IPTG for 4 hours. RNA was extracted, and Northern blots were performed using a probe designed to hybridize to one of the spacers in the crRNA array. A band pattern typical of crRNA processing was observed in the systems, with the strongest band corresponding to the single crRNA unit processed by Cash. Further maturation of crRNAs, probably reflecting 3′ end trimming, was also observed.

FIGS. 16A-B illustrates a fluorescence based system to measure RAMP activity. (FIG. 16A) Native spacers within the CRISPR array were replaced by spacers targeting GFP. A fourth plasmid (pRSF-Duet) was introduced to the system, expressing both GFP and RFP. (FIG. 16B) Expression of the Neisseria sicca RAMP system within E. coli, with spacers targeting GFP (red curve), results in reduction of GFP fluorescence but not of RFP, indicating an expression silencing at the RNA rather than the DNA level. No such reduction was observed when 4 native spacers were expressed in the crRNA (blue curve). Fluorescence and O.D. were measured every 13 minutes in biological quadruplicates for E. coli continuously growing with shaking at 37° C. within a robotic plate reader (Tecan Infinite 200 Pro).

FIG. 17 is a schematic representation of a construct based on the Neiserria sicca RAMP repeat-spacer array that allows the insertion of any spacer of choice.

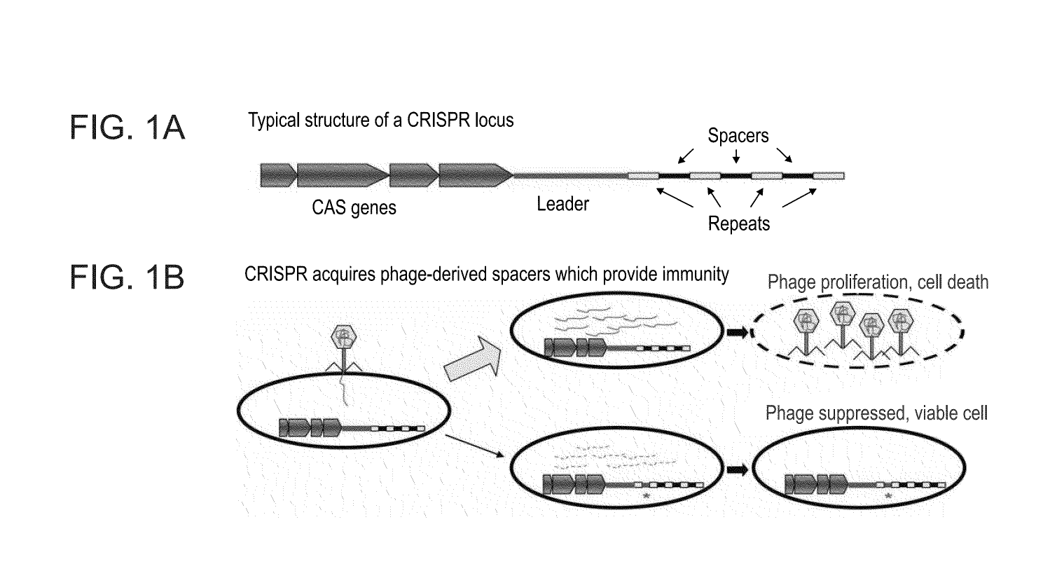

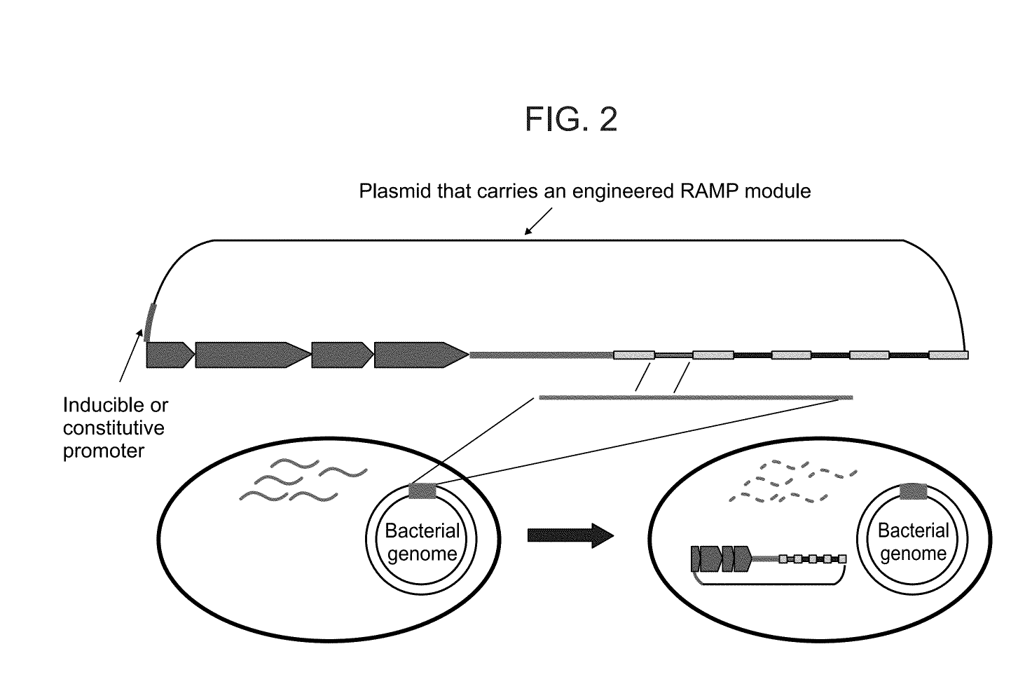

### DESCRIPTION OF SPECIFIC EMBODIMENTS OF THE INVENTION

The present invention, in some embodiments thereof, relates to methods of downregulating prokaryotic genes and, more particularly, but not exclusively, to methods of downregulating bacterial genes.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The clusters of regularly interspaced short palindromic repeat (CRISPR) system is associated with defense of bacteria and archaea providing protection thereto against invading phages. Resistance is acquired by incorporating short stretches of invading DNA sequences in genomic CRISPR loci. These integrated sequences are thought to function as a genetic memory that prevents the host from being infected by viruses containing this recognition sequence.

It has previously been proposed that CRISPR's ability to down-regulate extrachromosomal DNA may be manipulated such that it can also down-regulate genes that are integrated into the chromosome of a cell. Thus, it was proposed that in order to selectively down-regulate a gene of interest, a CRISPR-bearing plasmid may be transformed into a prokaryotic cell of choice (e.g. bacterial cell) with one of the spacers being changed to match the gene of interest (Sorek et al, 2008, Nature Reviews Microbiology, 6, 181).

However, previous attempts to engineer the CRISPR system to down regulate endogenous genes have failed, due to the discovery that many CRISPR systems target DNA rather than RNA [Marraffini & Sontheimer, Science 322, 1843 (2008)]. In order to circumvent this problem, the present inventors suggest using a specific CRISPR subtype, called the RAMP module (or cmr module). Recent evidence suggests that the RAMP module (or cmr module) is unique, and performs its action by silencing RNA rather than DNA [Hale et al, Cell 139, 863, 2009]. However, most natural RAMP modules, including the one that was experimentally examined [Hale et al, Cell 139, 863, 2009], occur within thermophilic (high-temperature-adapter) prokaryotes, and are thus not expected to work well in human pathogens and in model organisms such as E. coli.

The present inventors propose engineering polynucleotides of the CRISPR system which encode a CRISPR array and at least one CRISPR associated (CAS) polypeptide of a repeat associated mysterious protein (RAMP) family so that its spacers will target endogenous genes. This targeting is expected to result in degradation of the targeted mRNA, thus allowing selective silencing of specific genes of choice within bacteria, i.e., selective gene knock-down without manipulation of the original microbial genome (as explained above).

Thus, according to one aspect of the present invention there is provided a method of down-regulating expression of a gene of a prokaryotic cell. The method comprises introducing into the cell a CRISPR system encoding a CRISPR array and at least one CRISPR associated (CAS) polypeptide of a repeat associated mysterious protein (RAMP) family, wherein a spacer of the CRISPR array is sufficiently complementary with a portion of the gene to down-regulate expression of the gene.

In an exemplary embodiment, the gene is not introduced into the cell by a bacteriophage.

As used herein, the phrase “down-regulating” refers to reducing or inhibiting the expression level of the gene, on the RNA and optionally on the protein level. According to one embodiment, the gene is down-regulated by at least 10%. According to another embodiment, the gene is down-regulated by at least 20%. According to another embodiment, the gene is down-regulated by at least 30%. According to another embodiment, the gene is down-regulated by at least 40%. According to another embodiment, the gene is down-regulated by at least 50%. According to another embodiment, the gene is down-regulated by at least 60%. According to another embodiment, the gene is down-regulated by at least 70%. According to another embodiment, the gene is down-regulated by at least 80%. According to another embodiment, the gene is down-regulated by at least 90%. According to another embodiment, the gene is down-regulated by 100% (i.e. inhibiting gene expression).

As used herein, the term “gene” refers to a DNA sequence which encodes a polypeptide or a non-coding, functional RNA.

The phrase “gene of a prokaryotic cell” refers to a gene that is present in the prokaryotic cell but not necessarily integrated into the chromosome of the prokaryotic cell.

According to one embodiment, the genes which are down-regulated are those that are integrated into the chromosome of the prokaryote.

According to another embodiment, the genes which are down-regulated are those that remain outside the chromosome i.e. remain epichromosomal.

According to another embodiment, the gene is endogenous to the cell. The term “endogenous gene” refers to a native gene in its natural location in the genome of an organism.

Examples of contemplated genes that may be downregulated according to the method of this aspect of the present invention, include, but are not limited to genes associated with down-regulation of organic material production in bacteria.

Thus, for example, the present invention contemplates the down-regulation of genes whose knockout enhanced the production of ethanol as a biofuel. Shaw et al., (PNAS 2008, Sep. 16; 105(37):1769-74) teaches the knock-out of a number of genes (namely acetate kinase, phosphate acetyltransferase and L-lactate dehydrogenase, examples of sequences of each can be found in refseq accession no: NC_{—}009012, Clostridium thermocellum ATCC 27405, complete genome) that resulted in the production of ethanol at high yields.

Tannler S et al [Metab Eng. 2008 September; 10(5):216-26], teaches enhanced ethanol production in bacteria by down-regulating expression of the gluconeogenic genes gapB and pckA (examples of sequences of each can be found in refseq accession no: NC_{—}000964, Bacillus subtilis subsp. subtilis str. 168, complete genome) through knockout of their genetic repressor CcpN.

The present invention also contemplates the down-regulation of genes whose knockout enhanced the production of hydrogen as a biofuel.

Vardar-Schara et al [Microbial Biotechnology Vol 1, Issue 2, Pages 107-125], incorporated herein by reference, teaches a number of strains of genetically engineered bacteria which generate hydrogen. Vardar-Schara et al states therein, that the hydrogen yield is suboptimal in a number of those strains due to the presence of uptake hydrogenases. Accordingly, the present invention contemplates downregulation of these uptake hydrogenases, Hyd-1 and -2 (hyaB and hybC respectively) for the enhancement of hydrogen production. Examples of sequences of each can be found in refseq accession no: NC_{—}011742, Escherichia coli S88, complete genome.

In addition, the present invention contemplates down-regulation of genes of metabolic pathways that compete for hydrogen production.

Exemplary genes that may be down-regulated to increase hydrogen production in bacteria include, but are not limited to lactate dehydrogenase (ldhA), the FHL repressor (hycA), fumarate reductase (frdBC), the Tat system (tatA-E), the alpha subunit of the formate dehydrogenase-N and -O (fdnG and fdoG respectively), the alpha subunit of nitrate reductase A (narG), pyruvate dehydrogenase (aceE), pyruvate oxidase (poxB) and proteins that transport formate (focA and focB)—see Vardar-Schara et al [Microbial Biotechnology Vol 1, Issue 2, Pages 107-125]. Examples of sequences of each can be found in refseq accession no: NC_{—}011742, Escherichia coli S88, complete genome.

Lactic Acid Bacteria (LAB) play an essential role in the preservation, taste and texture of cheese, yogurt, sausage, sauerkraut and a large variety of traditional indigenous fermented foods. Down-regulation of such genes would ensure for example that lactic acid bacteria used in the food industry would have a better taste or smell. According to another embodiment, the genes that are down-regulated in bacteria are those which are involved in taste or odor.

For example, the buttermilk aroma diacetyl is formed from the carbon metabolism of dairy Lactococcus bacteria during buttermilk fermentation. Lactococcal strains that have low levels of a diacetyl reductase, acetoin reductase and butanediol dehydrogenase have been found to produce more diacetyl. Down-regulation of such enzymes would therefore be beneficial. Examples of sequences of each can be found in refseq accession no: NC_{—}009004, Lactococcus lactis subsp. cremoris MG1363.

For example, down-regulation of those Lactobacillus bulgaricus genes associated with lactic acid production would be beneficial for the generation of mild forms of yoghurt.

As used herein, the expression “lactic acid bacterium” refers to a group of gram-positive, microaerophilic or anaerobic bacteria having in common the ability to ferment sugars and citrate with the production of acids including lactic acid as the predominantly produced acid, acetic acid, formic acid and propionic acid. The industrially most useful lactic acid bacteria are found among Lactococcus species, Streptococcus species, Lactobacillus species, Leuconostoc species, Oenococcus species and Pediococcus species. In the dairy industry, the strict anaerobes belonging to the genus Bifidobacterium is generally included in the group of lactic acid bacteria as these organisms also produce lactic acid and are used as starter cultures in the production of dairy products.

It will be appreciated that the present invention may be used to enhance production of any industrial, agricultural, pharmaceutical (e.g. recombinant protein production) product in bacteria by suppressing genes associated with lower levels of expression of that industrial product.

Other examples of bacterial genes that may be downregulated according to the method of this aspect of the present invention are genes that if down-regulated would aid in the treatment of a bacterial infection. Such genes include for example, antibiotic resistance genes, bacterial virulence genes and genes that are essential for the growth of bacteria.

The phrase “antibiotic resistance genes” as used herein refers to genes that confer resistance to antibiotics, for example by coding for enzymes which destroy it, by coding for surface proteins which prevent it from entering the microorganism, or by being a mutant form of the antibiotic's target so that it can ignore it.

Example of antibiotic resistance genes may be found on the ARDB—Antibiotic Resistance Genes Database—www.ardbdotcbcbdotumddotedu/. Particular examples of antibiotic resistance genes include, but are not limited to methicillin resistance gene or a vancomycin resistance gene.

The phrase “virulence gene” as used herein refers to a nucleic acid sequence of a microorganism, the presence and/or expression of which correlates with the pathogenicity of the microorganism. In the case of bacteria, such virulence genes may in an embodiment comprise chromosomal genes (i.e. derived from a bacterial chromosome), or in a further embodiment comprise a non-chromosomal gene (i.e. derived from a bacterial non-chromosomal nucleic acid source, such as a plasmid). In the case of E. coli, examples of virulence genes and classes of polypeptides encoded by such genes are described below. Virulence genes for a variety of pathogenic microorganisms are known in the art.

Examples of virulence genes include, but are not limited to genes encoding toxins, hemolysins, fimbrial and afimbrial adhesins, cytotoxic factors, microcins and colicins and also those identified in Sun et al., Nature medicine, 2000; 6(11): 1269-1273.

According to one embodiment of the invention, the bacterial virulence gene may be selected from the group consisting of actA (example is given in genebank accession no: NC_{—}003210.1), Tem (example is given in genebank accession no: NC_{—}009980), Shv (example is given in genebank accession no: NC_{—}009648), oxa-1 (example is given in genebank accession no: NW_{—}139440), oxa-7 (example is given in genebank accession no: X75562), pse-4 (example is given in genebank accession no: J05162), ctx-m (example is given in genebank accession no: NC_{—}010870), ant(3″)-Ia (aadA1) (example is given in genebank accession no: DQ489717), ant(2″)-Ia (aadB)b (example is given in genebank accession no: DQ176450), aac(3)-IIa (aacC2) (example is given in genebank accession no: NC_{—}010886), aac(3)-IV (example is given in genebank accession no: DQ241380), aph(3′)-Ia (aphA1) (example is given in genebank accession no: NC_{—}007682), aph(3′)-IIa (aphA2) (example is given in genebank accession no: NC_{—}010170), tet(A) (example is given in genebank accession no: NC_{—}005327), tet(B) (example is given in genebank accession no: FJ411076), tet(C) (example is given in genebank accession no: NC_{—}010558), tet(D) (example is given in genebank accession no: NC_{—}010558), tet(E) (example is given in genebank accession no: M34933), tet(Y) (example is given in genebank accession no: AB089608), catI (example is given in genebank accession no: NC_{—}005773), catII NC_{—}010119, catIII (example is given in genebank accession no: X07848), floR (example is given in genebank accession no: NC_{—}009140), dhfrI (example is given in genebank accession no: NC_{—}002525), dhfrV (example is given in genebank accession no: NC_{—}010488), dhfrVII (example is given in genebank accession no: DQ388126), dhfrIX (example is given in genebank accession no: NC_{—}010410), dhfrXIII (example is given in genebank accession no: NC_{—}000962), dhfrXV (example is given in genebank accession no: Z83311), suII (example is given in genebank accession no: NC_{—}000913), suIII (example is given in genebank accession no: NC_{—}000913), integron class 1 3′-CS (example is given in genebank accession no: AJ867812), vat (example is given in genebank accession no: NC_{—}011742), vatC (example is given in genebank accession no: AF015628), vatD (example is given in genebank accession no: AF368302), vatE (example is given in genebank accession no: NC_{—}004566), vga (example is given in genebank accession no: AF117259), vgb (example is given in genebank accession no: AF117258), and vgbB (example is given in genebank accession no: AF015628).

As mentioned, in order to kill various pathogenic bacteria, CRISPR could be used in order to silence essential genes (i.e., compatible with life) in the bacteria. Essential genes could be identified by their conservation among several pathogens (Payne et al, Nature Reviews Drug Discovery 6, 29-40 (January 2007)). Such genes include ribosomal RNA genes (16S and 23S), ribosomal protein genes, tRNA-synthetases, as well as additional genes shown to be essential such as dnaB, fabI, folA, gyrB, murA, pytH, metG, and tufA(B) NC_{—}009641 for Staphylococcus aureus subsp. aureus str. Newman and NC_{—}003485 for Streptococcus pyogenes MGAS8232 (DeVito et al, Nature Biotechnology 20, 478-483 (2002)).

As mentioned, the present invention teaches a method of down-regulating a gene (or genes) in a prokaryotic cell.

Examples of prokaryotic cells include, but are not limited to bacterial cells and archaeal cells (e.g. those belonging to the two main phyla, the Euryarchaeota and Crenarchaeota).

The bacteria whose genes may be down-regulated may be gram positive or gram negative bacteria. The bacteria may also be photosynthetic bacteria (e.g. cyanobacteria).

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

The term “Gram-negative bacteria” as used herein refer to bacteria characterized by the presence of a double membrane surrounding each bacterial cell. Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica and Yersinia pestis.

As mentioned, the method of the present invention is effected by introducing into the cell a CRISPR system polynucleotide encoding a CRISPR array and at least one CRISPR associated (CAS) polypeptide of a repeat associated mysterious protein (RAMP) family, wherein a spacer of the CRISPR array is sufficiently complementary with a portion of the gene to down-regulate expression of the gene.

According to another embodiment the method is effected by introducing into the cell a first polynucleotide which encodes the CRISPR array and a second polynucleotide which encodes at least one Cas polypeptide of the RAMP family.

Below is a short summary of CRISPR arrays.

CRISPR (Clustered Regularly Interspaced Short Palindromic Repeats) arrays together with the CAS genes form the CRISPR system.

CRISPR arrays also known as SPIDRs (SPacer Interspersed Direct Repeats) constitute a family of recently described DNA loci that are usually specific to a particular bacterial species. The CRISPR array is a distinct class of interspersed short sequence repeats (SSRs) that were first recognized in E. coli (Ishino et al, J. Bacteriol., 169:5429-5433). In subsequent years, similar CRISPR arrays were found in Mycobacterium tuberculosis, Haloferax mediterranei, Methanocaldococcus jannaschii, Thermotoga maritima and other bacteria and archaea. The repeats of CRISPR arrays are short elements that occur in clusters that are always regularly spaced by unique intervening sequences with a constant length. Although the repeat sequences are highly conserved between strains, the number of interspersed repeats and the sequences of the spacer regions differ from strain to strain. The repeat sequences are partially palindromic DNA repeats typically of 24 to 47 bp, containing inner and terminal inverted repeats of up to 11 bp. These repeats have been reported to occur from 1 to 382 times. Although isolated elements have been detected, they are generally arranged in clusters (up to about 20 or more per genome) of repeated units spaced by unique intervening 26-72 bp sequences.

As used herein, the phrase “CRISPR array polynucleotide” refers to a DNA or RNA segment which comprises sufficient CRISPR repeats such that it is capable of downregulating a complementary gene.

According to one embodiment, the CRISPR array polynucleotide comprises at least 2 repeats with 1 spacer between them.

According to another embodiment, the CRISPR array polynucleotide comprises at least 4 repeats with spacers inbetween each.

According to still another embodiment, at least one, at least two, at least three, at least four of the spacers of the CRISPR array polynucleotide are the native sequences of the array.

According to one embodiment, the CRISPR array polynucleotide comprises at least 1 spacer flanked on the 5′ end by 4-10 bases from the 3′ end of the repeat.

In an exemplary embodiment, the CRISPR array polynucleotide comprises all of the CRISPR repeats, starting with the first nucleotide of the first CRISPR repeat and ending with the last nucleotide of the last (terminal) repeat.

Various computer software and web resources are available for the analysis of and identification of CRISPR systems and therefore CRISPR arrays. These tools include software for CRISPR detection, such as PILERCR, CRISPR Recognition Tool and CRISPRFinder; online repositories of pre-analyzed CRISPRs, such as CRISPRdb; and tools for browsing CRISPRs in microbial genomes, such as Pygram. Databases for CRISPR systems include: www.crisprdotu-psuddotfr/crispr/CRISPRHomePagedotphp.

It has been revealed that CRISPR systems are found in approximately 40% and 90% of sequenced bacterial and archaeal genomes, respectively, and the present inventor contemplates the use of CRISPR arrays from all such CRISPR systems that target RNA.

According to one embodiment, the CRISPR array polynucleotide comprises a nucleic acid sequence which, apart from the spacer, (or spacers) which is replaced so as to down-regulate the gene of interest, is 100% homologous to the naturally occurring (wild-type) sequence.

According to another embodiment, the CRISPR array polynucleotide comprises a nucleic acid sequence which, apart from the spacer, (or spacers) which is replaced so as to down-regulate a gene of interest, is 99% homologous to the naturally occurring (wild-type) sequence.

According to another embodiment, the CRISPR array polynucleotide comprises a nucleic acid sequence which, apart from the spacer, (or spacers) which is replaced so as to down-regulate a gene of interest, is 98% homologous to the naturally occurring (wild-type) sequence.

According to another embodiment, the CRISPR array polynucleotide comprises a nucleic acid sequence which, apart from the spacer, (or spacers) which is replaced so as to down-regulate a gene of interest, is 97% homologous to the naturally occurring (wild-type) sequence.

According to another embodiment, the CRISPR array polynucleotide comprises a nucleic acid sequence which, apart from the spacer, (or spacers) which is replaced so as to down-regulate a gene of interest, is 96% homologous to the naturally occurring (wild-type) sequence.

According to another embodiment, the CRISPR array polynucleotide comprises a nucleic acid sequence which, apart from the spacer, (or spacers) which is replaced so as to down-regulate a gene of interest, is 95% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 90% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 80% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 75% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 70% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 65% homologous to the naturally occurring (wild-type) sequence.

According to one embodiment, the CRISPR array polynucleotide comprises a nucleic acid sequence which, apart from the spacer, (or spacers) which is replaced so as to down-regulate a gene of interest, is 60% homologous to the naturally occurring (wild-type) sequence.

The present invention contemplates modification of the CRISPR system polynucleotide sequence such that the codon usage is optimized for the organism in which it is being introduced (e.g. E. coli).

Thus for example Cas6 polynucleotide sequence derived from Neisseria sicca codon optimized for use in E. coli is set forth in SEQ ID NO: 1361. A Cmr 1 polynucleotide sequence derived from Neisseria sicca codon optimized for use in E. coli is set forth in SEQ ID NO: 1362. A Cmr2 polynucleotide sequence derived from Neisseria sicca codon optimized for use in E. coli is set forth in SEQ ID NO: 1363. A Cmr3 polynucleotide sequence derived from Neisseria sicca codon optimized for use in E. coli is set forth in SEQ ID NO: 1364. A Cmr4 polynucleotide sequence derived from Neisseria sicca codon optimized for use in E. coli is set forth in SEQ ID NO: 1365. A Cmr5 polynucleotide sequence derived from Neisseria sicca codon optimized for use in E. coli is set forth in SEQ ID NO: 1366. A Cmr6 polynucleotide sequence derived from Neisseria sicca codon optimized for use in E. coli is set forth in SEQ ID NO: 1367.

Contemplated CRISPR systems that may be used according to this aspect of the present invention include, but are not limited to the CRISPR systems which encode at least one CRISPR associated (CAS) polypeptide of a repeat associated mysterious protein (RAMP) family.

As used herein, the term “cas gene” refers to the genes that are generally coupled, associated or close to or in the vicinity of flanking CRISPR arrays that encode CAS proteins.

According to one embodiment the cas gene is defined as such in one of the TIGRFAM profiles that were defined in [Haft et al, PLoS Comput Biol. 1, e60, 2005].

Preferably, a cas gene comprises at least 50%, more preferably at least 65%, more preferably at least 80%, more preferably at least 85%, more preferably at least 90%, more preferably at least 95%, more preferably at least 96%, more preferably at least 97%, more preferably at least 98%, most preferably at least 99% of the wild-type sequence. Preferably, a cas gene retains 50%, more preferably 60%, more preferably 70%, more preferably 80%, more preferably 85%, more preferably 90%, more preferably 95%, more preferably 96%, more preferably 97%, more preferably 98%, or most preferably 99% activity of the wild-type polypeptide or nucleotide sequence.

CRISPR arrays are typically found in the vicinity of four genes named cas1 to cas4. The most common arrangement of these genes is cas3-cas4-cas 1-cas2. The Cas3 protein appears to be a helicase, whereas Cas4 resembles the RecB family of exonucleases and contains a cysteine-rich motif, suggestive of DNA binding. The cas1 gene (NCBI COGs database code: COG1518) is especially noteworthy, as it serves as a universal marker of the CRISPR system (linked to all CRISPR systems except for that of Pyrococcus abyssii). Cas2 remains to be characterized cas1-4 are typically characterized by their close proximity to the CRISPR loci and their broad distribution across bacterial and archaeal species. Although not all cas1-4 genes associate with all CRISPR loci, they are all found in multiple subtypes.

In addition, there is another cluster of three genes associated with CRISPR structures in many bacterial species, referred to herein as cas1 B, cas5 and cas6; (See, [Barrangou et al, 2007, Science 315(5819): 1709-12]). In some embodiments, the cas gene is selected from cas1, cas2, cas3, cas4, cas IB, cas5 and/or cas6, fragments, variants, homologues and/or derivatives thereof. In some additional embodiments, a combination of two or more cas genes find use, including any suitable combinations, including those provided in WO 07/025097, incorporated herein by reference.

In some embodiments, the cas genes comprise DNA, while in other embodiments, the cas comprise RNA. In some embodiments, the nucleic acid is of genomic origin, while in other embodiments, it is of synthetic or recombinant origin. In some embodiments, the cas genes are double-stranded or single-stranded whether representing the sense or antisense strand or combinations thereof.

In some embodiments it is preferred that the cas gene is the cas gene that is closest to the leader sequence or the first CRISPR repeat at the 5′ end of the CRISPR locus—such as cas4 or cas6.

Exemplary CAS polypeptides of the RAMP subtype include Csm3-5, Cmr1, Cmr2, Cmr3, Cmr4, Cmr6 and Csx7.

Exemplary cas gene sequences of the RAMP subtype are set forth in SEQ ID NOs: 1-1339.

According to a specific embodiment, the CRISPR system encodes at least a Cas6 and the six gene operon Cmr1-Cmr6 of the Neisseria sicca bacteria. The CRISPR system may further encode Cas1 and Cas2 of the Neisseria sicca bacteria.

According to one embodiment, the CRISPR system polynucleotide sequence encodes at least 2, at least 3, at least 4, at least 5, at least 6, at least 7 or all the CAS polypeptides of the RAMP family.

According to another embodiment, the CRISPR system polynucleotide sequence encodes a combination of cas polypeptides that is found to be naturally occurring, wherein at least one cas polypeptide belongs to the RAMP subtype. Such a combination is referred to herein as a RAMP module.

Examples of CRISPR systems of the RAMP module are detailed in Table 1 in the Examples section herein below.

It will be appreciated that a given set of cas genes or proteins is typically associated with a given repeated sequence within a particular CRISPR array. Thus, cas genes appear to be specific for a given DNA repeat (i.e., cas genes and the repeated sequence form a functional pair).

Thus, for example if the CRISPR array which is used to downregulate a gene comprises the same repeat sequences as the CRISPR array from the Myxococcus xanthus DK 1622 RAMP module CRISPR system, then the cas genes that may also be comprised in the polynucleotide may be those from the Myxococcus xanthus DK 1622 CRISPR system. According to one embodiment, the same number of cas genes are added to the polynucleotide as the number of cas genes that appear in the original system. According to another embodiment, at least one of the cas genes that appears in the original system is added to the polynucleotide.

Thus for example, cas genes that appear in the Myxococcus xanthus DK 1622 RAMP module CRISPR system include Cas6 (SEQ ID NO: 2), cmr6 SEQ ID NO: 4, cmr5 SEQ ID NO: 6, cmr4 SEQ ID NO: 8, cmr3 SEQ ID NO: 10, cmr2 SEQ ID NO: 12, cmr1 SEQ ID NO: 14, and a putative CRISPR-associated protein SEQ ID NO: 1338. Polynucleotides comprising repeat sequences from the Myxococcus xanthus DK 1622 CRISPR system may therefore preferably comprise at least one of these sequences.

As another example, Neisseria sicca Cas proteins include the Cas6 protein (SEQ ID NO: 1354), and the six Cmr1-Cmr6 proteins (SEQ ID NOs: 1355, 1356, 1357, 1358, 1359 and 1360). Polynucleotides comprising repeat sequences from Neisseria sicca (e.g. as set forth in SEQ ID NOs:1369, 1373, 1374 or 1375) and the leader sequence from Neisseria sicca (as set forth in SEQ ID NO: 1372) may therefore preferably comprise polynucleotide sequences encoding the above mentioned Cas proteins.

Typically, once a CRISPR system which encodes at least one cas protein of the RAMP module is identified it may be amplified and isolated.

Amplification of the CRISPR system may be achieved by any method known in the art, including polymerase chain reaction (PCR). In the present invention, oligonucleotide primers may be designed for use in PCR reactions to amplify all or part of a CRISPR array.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced (i.e., in the presence of nucleotides and an inducing agent—such as DNA polymerase and at a suitable temperature and pH). In some embodiments, the primer is single stranded for maximum efficiency in amplification, although in other embodiments, the primer is double stranded. In some embodiments, the primer is an oligodeoxyribonucleotide. The primer must be sufficiently long to prime the synthesis of extension products in the presence of the inducing agent. The exact length of the primers depends on many factors, including temperature, source of primer, and the use of the method. PCR primers are typically at least about 10 nucleotides in length, and most typically at least about 20 nucleotides in length. Methods for designing and conducting PCR are well known in the art, and include, but are not limited to methods using paired primers, nested primers, single specific primers, degenerate primers, gene-specific primers, vector-specific primers, partially mismatched primers, etc.

Exemplary primers that can amplify the M. xanthus CRISPR array are set forth in SEQ ID NO: 1350 and 1351.

As mentioned, in order to down-regulate the prokaryotic gene of interest, a spacer of the CRISPR array is replaced with a nucleic acid sequence, the nucleic acid sequence being sufficiently complementary to a portion of the prokaryotic gene.

As used herein, the term “spacer” refers to a non-repetitive spacer sequence that is found between multiple short direct repeats (i.e., CRISPR repeats) of CRISPR arrays. In some preferred embodiments, CRISPR spacers are located in between two identical CRISPR repeats. In some embodiments, CRISPR spacers are located in between two partial repeats. In some embodiments, CRISPR spacers are identified by sequence analysis at the DNA stretches located in between two CRISPR repeats.

In some preferred embodiments, CRISPR spacer is naturally present in between two identical, short direct repeats that are palindromic.

The phrase “portion of a gene” relates to a portion from the coding or non-coding region of the gene.

The phrase “sufficiently complementary” as used herein, refers to the sequence of the spacer being adequately complementary such that it is capable of downregulating expression of the gene.

According to one embodiment of this aspect of the present invention, a sequence which is sufficiently complementary to a portion of the prokaryotic gene is one which is at least about 70, about 75, about 80, about 85, or about 90% identical, or at least about 91, about 92, about 93, about 94, about 95, about 96, about 97, about 98, or about 99% identical to the prokaryotic gene. In some preferred embodiments, the sequence is 100% complementary to the prokaryotic gene.

Assays to test down-regulation of expression are known in the art and may be effected on the RNA or protein level.

Methods of Detecting the Expression Level of RNA

The expression level of the RNA in prokarytoic cells can be determined using methods known in the arts.

Northern Blot Analysis:

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR Analysis:

This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA In Situ Hybridization Stain:

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In Situ RT-PCR Stain:

This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide Microarray—

In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in prokaryotic cells can be determined using methods known in the arts.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to one embodiment, the spacer which is replaced has the same number of base pairs as the “replacing spacer” i.e. the one that is complementary to the prokaryotic gene.

According to another embodiment, at least two spacers of the CRISPR are replaced with a nucleic acid sequence, each nucleic acid sequence being sufficiently complementary to opposite strands of the gene.

The present inventor envisages that it is possible to replace any number of the spacers of the wild-type CRISPR.

The replacement spacers may target the same gene or a number of different genes. According to one embodiment, at least about 10% of the spacers are exchanged for a replacing spacer. According to another embodiment, at least about 20% of the spacers are exchanged for a replacing spacer. According to another embodiment, at least about 30% of the spacers are exchanged for a replacing spacer. According to another embodiment, at least about 40% of the spacers are exchanged for a replacing spacer. According to another embodiment, at least about 50% of the spacers are exchanged for a replacing spacer. According to another embodiment, at least about 60% of the spacers are exchanged for a replacing spacer. According to another embodiment, at least about 70% of the spacers are exchanged for a replacing spacer. According to another embodiment, at least about 80% of the spacers are exchanged for a replacing spacer. According to another embodiment, at least about 90% of the spacers are exchanged for a replacing spacer. According to still another embodiment, about 100% of the spacers are exchanged for a replacing spacer.

According to one embodiment, at least one of the replaced spacers in the CRISPR array of the present invention is the one which is at the most 5′ end of the array.

It will be appreciated that the polynucleotide which comprises the modified CRISPR array of the present invention may also comprise other sequences.

Thus, for example, the modified CRISPR array of the present invention may also comprise a leader sequence 5′ to the array.

The CRISPR leader is a conserved DNA segment of defined size which is located immediately upstream of the first repeat.

The leader sequence can be of a different length in different bacteria. In some embodiments, the leader sequence is at least about 20, about 25, about 30, about 35, about 40, about 45, about 50, about 55, about 60, about 65, about 70, about 75, about 80, about 85, about 90, about 95, about 100, about 200, about 300, about 400, or about 500 or more nucleotides in length.

According to one embodiment, the leader sequence is directly 5′ to the array with no intervening base pairs.

According to another embodiment the leader sequence is the same leader sequence found in the wild type CRISPR system.

Thus, for example if the CRISPR array that is introduced into the prokaryotic cell is derived from the Myxococcus xanthus DK 1622 CRISPR system of the RAMP module, then the leader sequence is that leader sequence that is found in the wild-type Myxococcus xanthus DK 1622 CRISPR system of the RAMP module.

Alternatively, if the CRISPR array that is introduced into the prokaryotic cell is derived from the Neisseria sicca CRISPR system of the RAMP module, then the leader sequence is that leader sequence that is found in the wild-type Neisseria sicca CRISPR system of the RAMP module (e.g. as set forth in SEQ ID NO: 1372).

According to an exemplary embodiment, where a prokaryotic cell already comprises a CRISPR system, the CRISPR array polynucleotide which is introduced into the cell comprises the identical CRISPR array repeat sequence which is endogenous to that bacteria (it does not necessarily have to have an array of the same size). Accordingly, the choice of CRISPR array that is introduced into a cell is mainly dependent on the prokaryote whose gene or genes are being down-regulated.

In the case where the prokaryotic cell does not comprise a CRISPR system it will be appreciated that any CRISPR array may be introduced into the cell. According to this embodiment, the other components which make up the CRISPR system are also introduced into the cell. Such components typically match the CRISPR array (i.e. originate from the same CRISPR system). The other components may be introduced into the cell (together with a non-modified, native spacer, or on their own) prior to administration of the CRISPR array with the modified spacer. Alternatively, the other components may be introduced into the cell concommitant with (on the same or on a separate vector) the CRISPR array with the modified spacer.

Typically, the polynucleotides of the present invention are inserted into nucleic acid constructs so that they are capable of being expressed and propagated in bacterial cells.

Such nucleic acid constructs typically comprise a prokaryotic origin of replication and other elements which drive the expression of the CRISPR array and associated cas genes.

Preferably, the promoter utilized by the nucleic acid construct of the present invention is active in the specific cell population transformed.

Constitutive promoters suitable for use with the present invention are promoter sequences which are active under most environmental conditions and most types of cells such as the cytomegalovirus (CMV) and Rous sarcoma virus (RSV).

According to one embodiment, the promoter is an inducible promoter, i.e., a promoter that induces the CRISPR expression only in a certain condition (e.g. heat-induced promoter) or in the presence of a certain substance (e.g., promoters induced by Arabinose, Lactose, IPTG etc).

Examples of bacterial constructs include the pET series of E. coli expression vectors [Studier et al. (1990) Methods in Enzymol. 185:60-89).

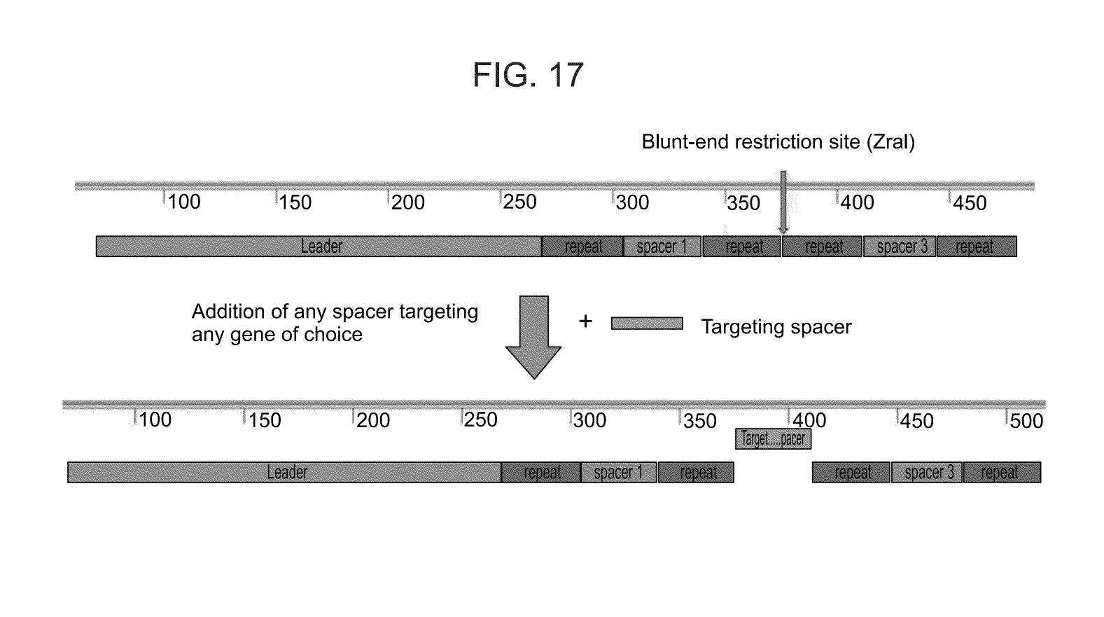

Additional nucleic acid constructs contemplated by the present inventors are those that are engineered such any spacer of choice can be inserted (using a simple blunt-end or sticky end ligation) between two repeat sequences (as exemplified in FIG. 17). This construct comprises at its minimum at least two repeats originating from a CRISPR system of the RAMP module. According to a specific embodiment the two repeats are concatenated (i.e. without an intermediate spacer sequence) which on joining make a unique restriction site there between. On cleavage, the insertion of any spacer to the CRISPR array may be effected using ligation.

The nucleic acid construct may optionally comprise a leader sequence upstream of the first repeat originating from the CRISPR system of the RAMP module.

In addition, the construct may comprise spacer sequences on one or both sides of the repeat sequences, as illustrated in FIG. 17.

Methods of introducing the polynucleotides of the present invention into prokaryotic cells are well known in the art—these include, but are not limited to, transforming with a recombinant bacteriophage DNA, plasmid DNA or cosmid DNA expression vector containing the CRISPR array sequence.

It will be appreciated that downregulating bacterial genes that are essential or vital to bacterial functioning may be used as a method for treating a bacterial infection. Similarly, downregulating bacterial genes that are associated with bacterial virulence may also be used as a method for treating a bacterial infection.

Thus, according to another aspect of the present invention, there is provided a method of treating a bacterial infection in a subject in need thereof, the method comprising administering to the subject a therapeutically effective amount of an isolated polynucleotide, comprising a clustered, regularly interspaced short palindromic repeat (CRISPR) array nucleic acid sequence wherein at least one spacer of the CRISPR is sufficiently complementary to a portion of at least one bacterial gene so as to down-regulate expression of the bacterial gene, the bacterial gene being a vital bacterial gene or a bacterial virulence gene, thereby treating the bacterial infection.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “bacterial infection” as used herein, refers the invasion and colonization of bacteria in a bodily tissue producing subsequent tissue injury and disease.

The bacterial infection may be on the body surface, localized (e.g., contained within an organ, at a site of a surgical wound or other wound, within an abscess), or may be systemic (e.g., the subject is bacteremic, e.g., suffers from sepsis). Of particular interest is the treatment of bacterial infections that are amenable to therapy by topical application of the phage of the invention. Also of particular interest is the treatment of bacterial infections that are present in an abscess or are otherwise contained at a site to which the phage of the invention can be administered directly.

The present invention also contemplates coating of surfaces other than body surfaces with the constructs of the present invention. U.S. Pat. No. 6,627,215 teaches coating of wound dressings with nucleic acids that comprise anti-bacterial activity. U.S. Pat. No. 6,617,142 teaches methods for attaching DNA or RNA to medical device surfaces.

Contacting a surface with the constructs can be effected using any method known in the art including spraying, spreading, wetting, immersing, dipping, painting, ultrasonic welding, welding, bonding or adhering. The peptides of the present invention may be attached as monolayers or multiple layers.

The present invention coating a wide variety of surfaces with the constructs of the present invention including fabrics, fibers, foams, films, concretes, masonries, glass, metals, plastics, polymers, and like.

An exemplary solid surface that may be coated with the peptides of the present invention is an intracorporial or extra-corporial medical device or implant.

An “implant” as used herein refers to any object intended for placement in a human body that is not a living tissue. The implant may be temporary or permanent. Implants include naturally derived objects that have been processed so that their living tissues have been devitalized. As an example, bone grafts can be processed so that their living cells are removed (acellularized), but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components.

Thus, for example, the present invention therefore envisions coating vascular stents with the peptides of the present invention. Another possible application of the peptides of the present invention is the coating of surfaces found in the medical and dental environment.

Surfaces found in medical environments include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; blood filters, implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherably insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, aneurysm repair devices, heart valves, artificial joints, artificial larynxes, otological implants), anastomotic devices, vascular catheter ports, clamps, embolic devices, wound drain tubes, hydrocephalus shunts, pacemakers and implantable defibrillators, and the like. Other examples will be readily apparent to practitioners in these arts.

Surfaces found in the medical environment include also the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilized drugs in nebulizers and of anesthetic agents. Also included are those surfaces intended as biological barriers to infectious organisms in medical settings, such as gloves, aprons and faceshields. Commonly used materials for biological barriers may be latex-based or non-latex based. Vinyl is commonly used as a material for non-latex surgical gloves. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered. Other surfaces include medical gauzes and plasters such as band-aids.

Other surfaces related to health include the inner and outer aspects of those articles involved in water purification, water storage and water delivery, and those articles involved in food processing. Thus the present invention envisions coating a solid surface of a food or beverage container to extend the shelf life of its contents.

Surfaces related to health can also include the inner and outer aspects of those household articles involved in providing for nutrition, sanitation or disease prevention. Examples can include food processing equipment for home use, materials for infant care, tampons and toilet bowls.

Typically, the subject being treated is a mammalian subject—e.g. human, fowl, rodent or primate.

According to one embodiment, the above-mentioned nucleic acid construct is administered as naked DNA or in a carrier—such as a liposome. According to another embodiment of this aspect of the present invention the polynucleotides are delivered to the bacteria using a targeting moiety (see for example Yacoby and Benhar, Infect Disord Drug Targets. 2007 September; 7(3):221-9).

Thus, according to another embodiment of this aspect of the present invention, the subject is administered with the polynucleotides of the present invention using bacteriophages. According to one embodiment, the bacteriophages are lytic phages.

Treatment of bacterial infections using bacteriophages is well known in the art.

Bacteriophage(s) suitable for use in treatment of a subject can be selected based upon the suspected bacterial pathogen infecting the subject. Methods for diagnosis of bacterial infections and determination of their sensitivities are well known in the art. Where such diagnosis involves culturing a biological sample from the subject, the clinician can at the same time test the susceptibility of the infecting pathogen to growth inhibition by one or more therapeutic phages that are candidates for subsequent therapy.

In order to address the problem of rapid clearance by the spleen, liver and the reticulo-endothelial system of bacteriophages, the present inventors contemplate the use of long-circulating variants of wild type phages (see for example Merrill et al (Proc. Natl. Acad. Sci. USA 93, 3188 (1996) and U.S. Pat. No. 5,688,501) or holing modified bacteriophages—see for example U.S. Pat. Appl. 20040156831.

Selection of the gene to be downregulated will depend on the bacterial infection being treated. According to one embodiment, the spacers of the modified CRISPRs of the present invention are designed such that they target a gene that is highly conserved among bacteria; such spacers will lead to broad-spectrum killing. According to one embodiment, the spacers of the modified CRISPRs of the present invention are designed such that they target a gene that is unique to a specific bacteria; such spacers will lead to narrow-spectrum killing.

According to another embodiment, the bacterial infection being treated is an antibiotic resistant bacterial infection—e.g. infections induced by methicillin resistant Staphylococcus aureus or vancomycin resistant Staphylococcus aureus. In this case, the spacers of the modified CRISPRs of the present invention are designed such that they target an antibiotic resistance gene.

The bacteriophages comprising the modified CRISPRs of the present invention may be administered per se, or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of the active agent to an organism.

Herein the term “active ingredient” refers to the modified CRISPR accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of bacterial infection or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As mentioned, the modified bacteria of the present invention may be present in food products as well as in food additives.

The phrase “food additive” [defined by the FDA in 21 C.F.R. 170.3(e)(1)] includes any liquid or solid material intended to be added to a food product. This material can, for example, include an agent having a distinct taste and/or flavor or a physiological effect (e.g., vitamins).

The food additive composition of the present invention can be added to a variety of food products.

As used herein, the phrase “food product” describes a material consisting essentially of protein, carbohydrate and/or fat, which is used in the body of an organism to sustain growth, repair and vital processes and to furnish energy. Food products may also contain supplementary substances such as minerals, vitamins and condiments. See Merriani-Webster's Collegiate Dictionary, 10th Edition, 1993. The phrase “food product” as used herein further includes a beverage adapted for human or animal consumption. A food product containing the food additive of the present invention can also include additional additives such as, for example, antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

It will be appreciated that the CRISPR polynucleotides of the present invention may also be used to identify a function of a particular prokaryotic gene.

According to this aspect of the present invention, the modified CRISPR polynucleotides of the present invention are introduced into prokaryotic cells, wherein a spacer of the CRISPR is directed against a prokaryotic gene of unknown function. A phenotype of the prokaryote is then assayed. Depending on the outcome of the assay, the function of the gene can be determined (i.e. annotated).

According to one embodiment, the phenotype is examined using “phenotype microarray analysis”—see for example Zhou et al Journal of Bacteriology, August 2003, p. 4956-4972, Vol. 185, No. 16.

The modified CRISPR arrays of the present invention may also be used to generate a library of clones, each of which containing a different down-regulated gene. Such libraries have been prepared for Escherichia coli K-12, [Baba et al., Construction of Escherichia coli K-12 in-frame, single-gene knockout mutants: the Keio collection” Molecular Systems Biology 2 Article number: 2006.0008]. However, construction of this library was extremely labor intensive and expensive. Organisms of interest for production of such libraries include bio-energy relevant organisms such as Synechocystis sp. PCC 6803 or in which it is needed to determine which genes are needed to be silenced in order to enhance the production of the desired output biofuel. This could also be done for organisms generating a biotechnologically relevant product (other than a bio-fuel). Organisms of interest for production of such libraries include human pathogens, animal pathogens and plant pathogens.

As mentioned, the constructs of the present invention may be useful in the generation of biofuels in bacteria.

Thus, according to another aspect of the present invention, there is provided a method of generating an organic material in bacteria, the method comprising downregulating a gene which compromises the generation of the organic material in the bacteria.

Using the CRISPR polynucleotides described herein, new virulence genes can be discovered in pathogens. According to this embodiment, a library of isolated polynucleotides, each comprising a clustered, regularly interspaced short palindromic repeat (CRISPR) array nucleic acid sequence wherein at least one spacer of the CRISPR is sufficiently complementary to a portion of at least one bacterial gene, will be constructed such that each isolated polynucleotide down regulates one gene in a specific pathogen. This library may then by used to serially down regulate each gene in the pathogen. Concomitant with down regulation of each of the genes separately, a virulence assay may be conducted, such that the virulence is measured when the specific gene is down regulated. Genes whose down regulation interferes with virulence will be identified as virulence-associated genes. Vaccines or antibiotics could be made to target these specific genes.

It is expected that during the life of a patent maturing from this application many relevant CRISPR arrays and systems will be identified and the scope of the term CRISPR array is intended to include all such new technologies a priori

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Identification and Characterization of RAMP Modules in Bacterial Genomes

Currently, there is no database or public resource that connects between CRISPR array and its associated cas genes. To identify RAMP modules in prokaryotes that can function in downregulating prokaryotic genes, the present inventors computationally scanned the genomes of 334 CRISPR-bearing organisms. Non-questionable CRISPR arrays, spacers, and repeats were obtained from CRISPRdb [Grissa et al, BMC Bioinformatics 8, 172, 2007]. For each CRISPR-bearing organism, information regarding cas genes was obtained from both the Genbank file and from IMG (Integrated Microbial Genomes) at JGI (www.imgdotjgidotdoedotgov/cgi-bin/pub/maindotcgi). For this example, cas genes were defined as genes that were assigned one of the TIGRFAM profiles that were defined in [Haft et al, PLoS Comput Biol. 1, e60, 2005]. For this example, an array of cas genes (cas array) was defined as one or more consecutive cas genes with up to 2 intervening non-cas genes. A “CRISPR system” was defined for this example as a cas array associated to its closest CRISPR array in the analyzed genome. RAMP modules were defined as a cas array+CRISPR array, where the cas array contains at least 4 genes, and where at least one gene belongs to the RAMP subtype, as defined by [Haft et al, PLoS Comput Biol. 1, e60, 2005].

Characterization of RAMP Modules in Bacterial Genomes:

The search above identified 73 RAMP modules (Table 1, herein below).

TABLE 1

| | NCBI | | Optimal | | cas_array_ | |
|---|---|---|---|---|---|---|
| No. | accession | organism name | growth temp | domain | accession | start pos |
| 1 | NC_008095 | Myxococcus xanthus | Mesophile | Bacteria | NC_008095.3 | 8888371 |
| | | DK 1622 | | | | |
| 2 | NC_002570 | Bacillus halodurans | Mesophile | Bacteria | NC_002570.1 | 347483 |
| | | C-125 | | | | |
| 3 | NC_010482 | Candidatus Korarchaecum | Mesophile | Archaea | NC_010482.2 | 433841 |
| | | cryptofilum OPF8 | | | | |
| 4 | NC_010803 | Chlorobium limicola | Mesophile | Bacteria | NC_010803.2 | 1009061 |
| | | DSM 245 | | | | |
| 5 | NC_011026 | Chloroherpeton thalassium | Mesophile | Bacteria | NC_011026.1 | 1118238 |
| | | ATCC 35110 | | | | |
| 6 | NC_009697 | Clostridium botulinum | Mesophile | Bacteria | NC_009697.2 | 2243224 |
| | | A ATCC 19397 | | | | |
| 7 | NC_009495 | Clostridium botulinum | Mesophile | Bacteria | NC_009495.2 | 2314499 |
| | | A ATCC 3502 | | | | |
| 8 | NC_009698 | Clostridium botulinum | Mesophile | Bacteria | NC_009698.2 | 2243443 |
| | | A Hall | | | | |
| 9 | NC_009699 | Clostridium botulinum | Mesophile | Bacteria | NC_009699.2 | 2365025 |
| | | F Langeland | | | | |
| 10 | NC_010546 | Cyanothece sp. | Mesophile | Bacteria | NC_010546.1 | 345339 |
| | | ATCC 51142 | | | | |
| 11 | NC_008789 | Halorhodospira halophila | Mesophile | Bacteria | NC_008789.1 | 697188 |
| | | SL1 | | | | |
| 12 | NC_009972 | Herpetosiphon aurantiacus | Mesophile | Bacteria | NC_009972.1 | 661191 |
| | | ATCC 23779 | | | | |
| 13 | NC_009972 | Herpetosiphon aurantiacus | Mesophile | Bacteria | NC_009972.2 | 798867 |
| | | ATCC 23779 | | | | |
| 14 | NC_009654 | Marinomonas sp. | Mesophile | Bacteria | NC_009654.1 | 3037434 |
| | | MWYL1 | | | | |
| 15 | NC_009634 | Methanococcus vannielii | Mesophile | Archaea | NC_009634.2 | 117348 |
| | | SB | | | | |
| 16 | NC_007355 | Methanosarcina barkeri | Mesophile | Archaca | NC_007355.2 | 1667328 |
| | | fusaro | | | | |
| 17 | NC_003901 | Methanosarcina mazei | Mesophile | Archaea | NC_003901.3 | 4081676 |
| | | Go1 | | | | |
| 18 | NC_003272 | Nostoc sp. PCC 7120 | Mesophile | Bacteria | NC_003272.3 | 1737678 |
| 19 | NC_010729 | Porphyromonas gingivalis | Mesophile | Bacteria | NC_010729.1 | 2146958 |
| | | ATCC 33277 | | | | |
| 20 | NC_002950 | Porphyromonas gingivalis | Mesophile | Bacteria | NC_002950.1 | 2069305 |
| | | W83 | | | | |
| 21 | NC_011059 | Prosthecochloris aestuarii | Mesophile | Bacteria | NC_011059.3 | 2176063 |
| | | SK413, DSM 271 | | | | |
| 22 | NC_010162 | Sorangium cellulosum | Mesophile | Bacteria | NC_010162.3 | 7992067 |
| | | So ce 56 | | | | |
| 23 | NC_008532 | Streptococcus thermophilus | Mesophile | Bacteria | NC_008532.2 | 895636 |
| | | LMD-9 | | | | |
| 24 | NC_006448 | Streptococcus thermophilus | Mesophile | Bacteria | NC_006448.2 | 862566 |
| | | LMG 18311 | | | | |
| 25 | NC_010480 | Synechococcus sp. | Mesophile | Bacteria | NC_010480.1 | 44557 |
| | | PCC 7002 | | | | |
| 26 | NC_005230 | Synechocystis sp. | Mesophile | Bacteria | NC_005230.4 | 80203 |
| | | PCC 6803 | | | | |
| 27 | NC_007759 | Syntrophus aciditrophicus | Mesophile | Bacteria | NC_007759.1 | 589475 |
| | | SB | | | | |
| 28 | NC_000918 | Aquifex aeolicus VF5 | Thermophile | Bacteria | NC_000918.2 | 245338 |
| 29 | NC_000917 | Archaeoglobus fulgidus | Thermophile | Archaea | NC_000917.2 | 1671367 |
| | | DSM 4304 | | | | |
| 30 | NC_009954 | Caldivirga maquilingensis | Thermophile | Archaea | NC_009954.1 | 1589302 |
| | | IC-167 | | | | |
| 31 | NC_010424 | Candidatus Desulforudis | Thermophile | Bacteria | NC_010424.2 | 1887334 |
| | | audaxviator MP104C | | | | |
| 32 | NC_010424 | Candidatus Desulforudis | Thermophile | Bacteria | NC_010424.3 | 1914037 |
| | | audaxviator MP104C | | | | |
| 33 | NC_010175 | Chloroflexus aurantiacus | Thermophile | Bacteria | NC_010175.5 | 3122962 |
| | | J-10-fl | | | | |
| 34 | NC_008025 | Deinococcus geothermalis | Thermophile | Bacteria | NC_008025.2 | 1025204 |
| | | DSM 11300 | | | | |
| 35 | NC_008818 | Hyperthermus butylicus | Thermophile | Archaea | NC_008818.3 | 688803 |
| | | DSM 5456 | | | | |
| 36 | NC_009776 | Ignicoccus hospitalis | Thermophile | Archaea | NC_009776.1 | 282698 |
| | | KIN4/I | | | | |
| 37 | NC_003551 | Methanopyrus kandleri | Thermophile | Archaea | NC_003551.2 | 1292953 |
| | | AV19 | | | | |
| 38 | NC_008553 | Methanosaeta thermophila | Thermophile | Archaea | NC_008553.1 | 662154 |
| | | PT | | | | |
| 39 | NC_000916 | Methanothermobacter | Thermophile | Archaea | NC_000916.1 | 259228 |
| | | thermautotrophicus Delta H | | | | |
| 40 | NC_009454 | Pelotomaculum | Thermophile | Bacteria | NC_009454.1 | 702243 |
| | | thermopropionicum SI | | | | |
| 41 | NC_009454 | Pelotomaculum | Thermophile | Bacteria | NC_009454.2 | 2026733 |
| | | thermopropionicum SI | | | | |
| 42 | NC_009376 | Pyrobaculum arsenaticum | Thermophile | Archaea | NC_009376.3 | 1004361 |
| | | DSM 13514 | | | | |
| 43 | NC_009073 | Pyrobaculum calidifontis | Thermophile | Archaea | NC_009073.3 | 265427 |
| | | JCM 11548 | | | | |
| 44 | NC_009073 | Pyrobaculum calidifontis | Thermophile | Archaea | NC_009073.5 | 1176822 |
| | | JCM 11548 | | | | |
| 45 | NC_003413 | Pyrococcus furiosus | Thermophile | Archaea | NC_003413.4 | 1066025 |
| | | DSM 3638 | | | | |
| 46 | NC_009767 | Roseiflexus castenholzii | Thermophile | Bacteria | NC_009767.2 | 224482 |
| | | DSM 13941 | | | | |
| 47 | NC_009523 | Roseiflexus sp. RS-1 | Thermophile | Bacteria | NC_009523.1 | 465177 |
| 48 | NC_009523 | Roseiflexus sp. RS-1 | Thermophile | Bacteria | NC_009523.3 | 1754446 |
| 49 | NC_009523 | Roseiflexus sp. RS-1 | Thermophile | Bacteria | NC_009523.5 | 3213377 |
| 50 | NC_008148 | Rubrobacter xylanophilus | Thermophile | Bacteria | NC_008148.1 | 263398 |
| | | DSM 9941 | | | | |
| 51 | NC_002754 | Sulfolobus solfataricus P2 | Thermophile | Archaea | NC_002754.10 | 1799790 |
| 52 | NC_002754 | Sulfolobus solfataricus P2 | Thermophile | Archaea | NC_002754.5 | 1277348 |
| 53 | NC_002754 | Sulfolobus solfataricus P2 | Thermophile | Archaea | NC_002754.7 | 1365033 |
| 54 | NC_002754 | Sulfolobus solfataricus P2 | Thermophile | Archaea | NC_002754.8 | 1564763 |
| 55 | NC_003106 | Sulfolobus tokodaii 7 | Thermophile | Archaea | NC_003106.1 | 10108 |
| 56 | NC_003106 | Sulfolobus tokodaii 7 | Thermophile | Archaea | NC_003106.9 | 1985714 |
| 57 | NC_010730 | Sulfurihydrogenibium sp. | Thermophile | Bacteria | NC_010730.1 | 675839 |
| | | YO3AOP1 | | | | |
| 58 | NC_007776 | Synechococcus sp. | Thermophile | Bacteria | NC_007776.2 | 596531 |
| | | JA-2-3Ba(2-13) | | | | |
| 59 | NC_007776 | Synechococcus sp. | Thermophile | Bacteria | NC_007776.5 | 871945 |
| | | JA-2-3Ba(2-13) | | | | |
| 60 | NC_007775 | Synechococcus sp. JA-3-3Ab | Thermophile | Bacteria | NC_007775.4 | 881677 |
| 61 | NC_007775 | Synechococcus sp. JA-3-3Ab | Thermophile | Bacteria | NC_007775.5 | 2559707 |
| 62 | NC_003869 | Thermoanaerobacter | Thermophile | Bacteria | NC_003869.2 | 2523470 |
| | | tengcongensis MH4 | | | | |
| 63 | NC_007333 | Thermobifida fusca YX | Thermophile | Bacteria | NC_007333.1 | 1819567 |
| 64 | NC_008698 | Thermofilum pendens Hrk 5 | Thermophile | Archaea | NC_008698.3 | 1228909 |
| 65 | NC_008698 | Thermofilum pendens Hrk5 | Thermophile | Archaca | NC_008698.4 | 1261982 |
| 66 | NC_010525 | Thermoproteus neutrophilus | Thermophile | Archaea | NC_010525.3 | 514713 |
| | | V24Sta | | | | |
| 67 | NC_009616 | Thermosipho melanesiensis | Thermophile | Bacteria | NC_009616.3 | 1642545 |
| | | BI429 | | | | |
| 68 | NC_009828 | Thermotoga lettingae | Thermophile | Bacteria | NC_009828.2 | 1250942 |
| | | TMO | | | | |
| 69 | NC_000853 | Thermotoga maritima | Thermophile | Bacteria | NC_000853.1 | 1766027 |
| | | MSB8 | | | | |
| 70 | NC_010483 | Thermotoga sp. RQ2 | Thermophile | Bacteria | NC_010483.1 | 297079 |
| 71 | NC_010483 | Thermotoga sp. RQ2 | Thermophile | Bacteria | NC_010483.3 | 1064228 |
| 72 | NC_005838 | Thermus thermophilus | Thermophile | Bacteria | NC_005838.2 | 106247 |
| | | HB 27 | | | | |
| 73 | NC_006462 | Thermus thermophilus | Thermophile | Bacteria | NC_006462.2 | 151610 |
| | | HB8 | | | | |

| | | | | | | Distance | |
|---|---|---|---|---|---|---|---|
| | | | | associated | | to closest | |
| | | array | no of | CRISPR | | CRISPR | |
| No. | end pos | number | genes | arrays | Closest array | array | subtype |
| 1 | 8896616 | 3 | 8 | .NC_008095_13 | NC_008095_13 | 305 | .ramp |
| 2 | 353902 | 1 | 6 | .NC_002570_2 | NC_002570_2 | 396 | .ramp |
| 3 | 463588 | 2 | 22 | .NC_010482_2 | NC_010482_2 | 205 | .ramp. |
| | | | | | | | apern |
| 4 | 1023753 | 2 | 7 | .NC_010803_3 | NC_010803_3 | 874 | .csx.ramp |
| 5 | 1126262 | 1 | 7 | | NC_011026_3 | 47984 | .ramp |
| 6 | 2248370 | 2 | 5 | | NC_009697_8 | 4065 | .ramp |
| 7 | 2319645 | 2 | 5 | | NC_009495_8 | 4065 | .ramp |
| 8 | 2248589 | 2 | 5 | | NC_009698_8 | 4065 | .ramp |
| 9 | 2373694 | 2 | 8 | .NC_009699_12 | NC_009699_12 | 188 | .ramp |
| 10 | 355535 | 1 | 9 | .NC_010546_1 | NC_010546_1 | 264 | .ramp |
| 11 | 713310 | 1 | 16 | .NC_008789_1 | NC_008789_1 | 462 | .ramp |
| 12 | 667817 | 1 | 6 | | NC_009972_3 | 243154 | .ramp |
| 13 | 805983 | 2 | 6 | | NC_009972_6 | 234076 | .ramp |
| 14 | 3045414 | 1 | 8 | .NC_009654_2 | NC_009654_2 | 130 | .ramp. |
| | | | | | | | ypest |
| 15 | 122250 | 2 | 5 | | NC_009634_1 | 5174 | .ramp |
| 16 | 1673445 | 2 | 4 | | NC_007355_11 | 5707 | .ramp.csx |
| 17 | 4087769 | 3 | 4 | | NC_003901_10 | 1541 | .csx.ramp |
| 18 | 1745966 | 3 | 8 | | NC_003272_7 | 4357 | .csx.ramp. |
| | | | | | | | ramp2 |
| 19 | 2156418 | 1 | 9 | | NC_010729_10 | 17330 | .ramp |
| 20 | 2077522 | 1 | 7 | .NC_002950_5 | NC_002950_5 | 401 | .ramp |
| 21 | 2187002 | 3 | 7 | .NC_011059_3 | NC_011059_3 | 534 | .ramp.csx |
| 22 | 8000671 | 3 | 6 | | NC_010162_24 | 21468 | .ramp |
| 23 | 904257 | 2 | 9 | | NC_008532_4 | 1434 | .mtube. |
| | | | | | | | ramp |
| 24 | 871795 | 2 | 11 | | NC_006448_2 | 1433 | .ramp. |
| | | | | | | | mtube |
| 25 | 51898 | 1 | 5 | | NC_010480_1 | 2630 | .ramp |
| 26 | 89898 | 4 | 9 | .NC_005230_3 | NC_005230_3 | 207 | .ramp |
| 27 | 601507 | 1 | 10 | .NC_007759_2 | NC_007759_2 | 264 | .ramp |
| 28 | 262955 | 2 | 17 | .NC_000918_3 | NC_000918_3 | 177 | .tneap. |
| | | | | .NC_000918_2 | | | ramp.csx |
| 29 | 1690587 | 2 | 21 | .NC_000917_3 | NC_000917_3 | 343 | .csx.apern. |
| | | | | | | | ramp |
| 30 | 1610359 | 1 | 18 | .NC_009954_7 | NC_009954_7 | 26 | .ramp.apern |
| 31 | 1898008 | 2 | 10 | .NC_010424_4 | NC_010424_4 | 293 | .ramp2.csx |
| 32 | 1931439 | 3 | 17 | | NC_010424_6 | 1965 | .ramp |
| 33 | 3126296 | 5 | 4 | | NC_010175_17 | 130648 | .ramp2.csx |
| 34 | 1033572 | 2 | 8 | .NC_008025_4 | NC_008025_4 | 191 | .ramp |
| 35 | 699716 | 3 | 6 | | NC_008818_2 | 2658 | .ramp |
| 36 | 289665 | 1 | 4 | .NC_009776_3 | NC_009776_3 | 956 | .ramp |
| 37 | 1306332 | 2 | 9 | .NC_003551_3 | NC_003551_3 | 582 | .ramp |
| 38 | 687708 | 1 | 17 | | NC_008553_15 | 7637 | .ramp.csx |
| 39 | 264481 | 1 | 4 | | NC_000916_1 | 188868 | .ramp.csx |
| 40 | 709845 | 1 | 6 | | NC_009454_2 | 1279297 | .ramp.csx |
| 41 | 2044723 | 2 | 16 | .NC_009454_5 | NC_009454_5 | 176 | .ramp.hmari |
| 42 | 1030712 | 3 | 21 | | NC_009376_2 | 2866 | .ramp.apern |
| 43 | 275220 | 3 | 8 | .NC_009073_2 | NC_009073_2 | 223 | .ramp |
| 44 | 1201070 | 5 | 21 | | NC_009073_6 | 2281 | .apern.ramp |
| 45 | 1082264 | 4 | 15 | .NC_003413_7 | NC_003413_7 | 482 | .ramp.tneap |
| 46 | 229766 | 2 | 5 | | NC_009767_2 | 46105 | .ramp. |
| | | | | | | | ramp2.csx |
| 47 | 470489 | 1 | 5 | | NC_009523_2 | 259307 | .ramp.csx. |
| | | | | | | | ramp2 |
| 48 | 1760978 | 3 | 7 | | NC_009523_23 | 18445 | .ramp2.csx |
| 49 | 3221428 | 5 | 5 | | NC_009523_47 | 55325 | .ramp.csx |
| 50 | 281538 | 1 | 16 | .NC_008148_2 | NC_008148_2 | 159 | .hmari.ramp |
| 51 | 1807514 | 10 | 6 | | NC_002754_10 | 2258 | .ramp |
| 52 | 1280716 | 5 | 5 | | NC_002754_5 | 16437 | .ramp2 |
| 53 | 1368454 | 7 | 4 | | NC_002754_7 | 51888 | .ramp |
| 54 | 1571206 | 8 | 5 | | NC_002754_9 | 172801 | .ramp |
| 55 | 14279 | 1 | 5 | | NC_003106_1 | 11654 | .ramp2 |
| 56 | 1993250 | 9 | 6 | | NC_003106_4 | 213515 | .ramp |
| 57 | 681484 | 1 | 6 | | NC_010730_3 | 11598 | .ramp |
| 58 | 609307 | 2 | 10 | | NC_007776_4 | 4040 | .csx.ramp2 |
| 59 | 878468 | 5 | 4 | | NC_007776_5 | 4760 | .ramp |
| 60 | 897338 | 4 | 12 | | NC_007775_2 | 5004 | .ramp.csx. |
| | | | | | | | ramp2 |
| 61 | 2570837 | 5 | 10 | .NC_007775_10 | NC_007775_10 | 252 | .ramp |
| 62 | 2535850 | 2 | 9 | | NC_003869_3 | 1374 | .ramp |
| 63 | 1826460 | 1 | 6 | .NC_007333_8 | NC_007333_8 | 746 | .ramp |
| 64 | 1234237 | 3 | 4 | .NC_008698_2 | NC_008698_2 | 811 | .ramp2. |
| | | | | | | | mtube |
| 65 | 1279407 | 4 | 10 | | NC_008698_6 | 1340 | .csc.ramp |
| 66 | 520737 | 3 | 5 | | NC_010525_1 | 1004 | .ramp |
| 67 | 1650062 | 3 | 7 | .NC_009616_5 | NC_009616_5 | 918 | .ramp |
| 68 | 1259314 | 2 | 7 | | NC_009828_2 | 116357 | .ramp.csx |
| 69 | 1779740 | 1 | 12 | .NC_000853_8 | NC_000853_8 | 70 | .ramp.hmari |
| 70 | 304608 | 1 | 6 | | NC_010483_1 | 239575 | .csx. mtube. |
| | | | | | | | ramp |
| 71 | 1077979 | 3 | 13 | .NC_010483_5 | NC_010483_5 | 123 | .ramp.hmari |
| 72 | 112532 | 2 | 6 | | NC_005838_3 | 4627 | .ramp |
| 73 | 157896 | 2 | 6 | | NC_006462_6 | 4627 | .ramp |

Of these, 46 were in thermophilic organisms (organisms that optimally grow in high temperatures), and 27 in mesophiles (organisms optimally growing at a near body temperature). It is expected that proteins expressed in thermophiles are adapted to work in high temperatures, and might have a less optimal function when expressed in temperatures near 37° C. Therefore, in order to use RAMP modules from thermophiles, it can be hypothesized that specific amino acids should be changed in the cas genes to allow function at lower temperatures. This could be done in a process of molecular evolution.

The sequences of the 73 identified RAMP modules, as well as their associated repeat arrays and cas gene sequences, are set forth in SEQ ID NOs 1-1339).

### Example 2

### Down-Regulation of Three Genes in E. coli Bacteria

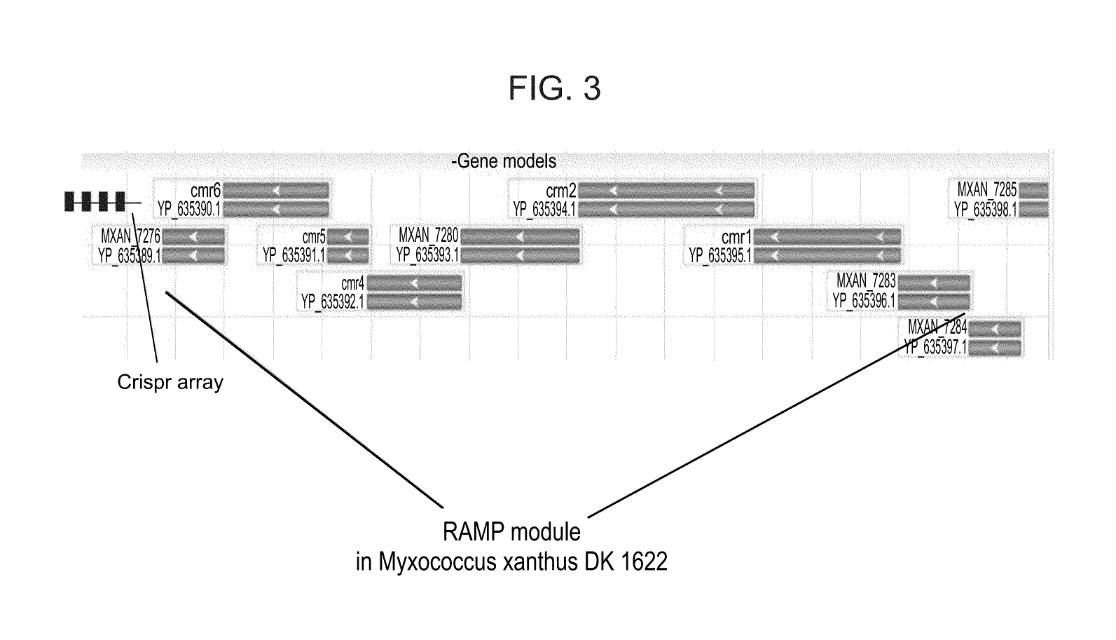

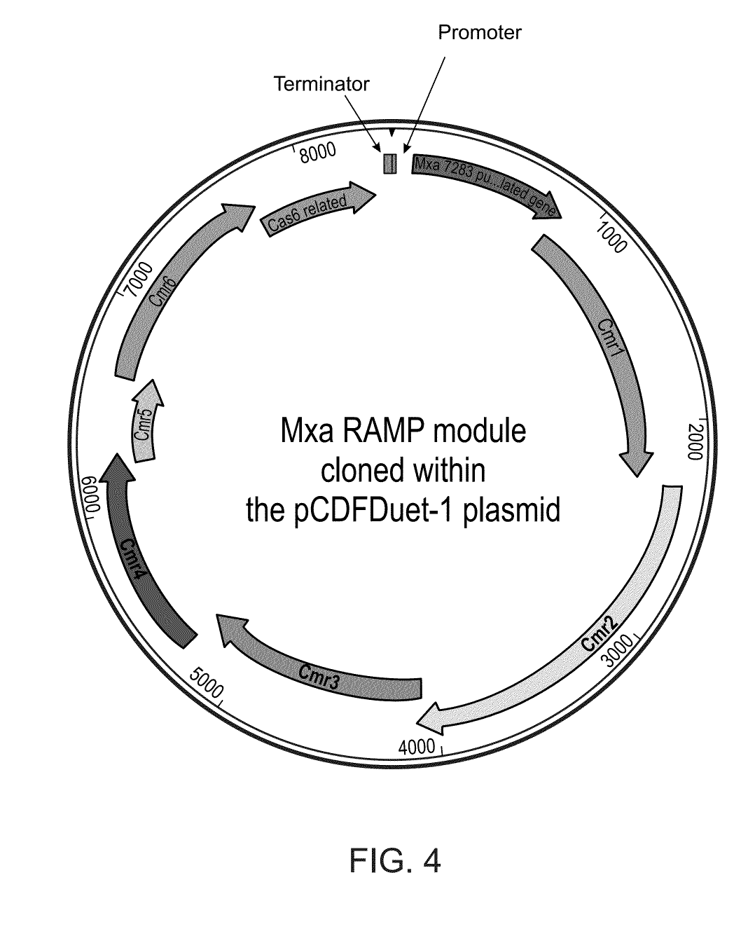

As a proof of concept, a RAMP module of the mesophilic organism Myxococcus xanthus DK 1622 may be used to silence three genes in E. coli. The RAMP module in this organism is found between positions 8888371-8896616 in its genome (NCBI accession NC_{—}008095), and contains 8 genes (FIG. 3). The three genes are GFP, RFP and malF (GFP and RFP are inserted into the E coli chromosome for the purpose of the proof of concept, while malF is a naturally occurring endogenous gene). Silencing of GFP and/or RFP is expected to result in loss of fluorescent emission, whist silencing of malF is expected to result in loss of ability to grow on maltose as the sole carbon source. In the present case, the spacers will target the cellular genes malF, GFP and RFP. In each construct four spacers will be designed targeting the antisense strand of each gene.

The cas array of the RAMP module will be amplified from the genome of Myxococcus xanthus DK 1622 using the following primers

AAAAAGATCTGATGAGACCACGAGGAGGTGATGTC (SEQ ID NO: 1350);

TTAATTAACACCGGCAAGCCTTCACGCGGCC (SEQ ID NO: 1351).

The amplified DNA will be cloned in a plasmid under the control of an inducible promoter and will be transformed to the E. coli strain BL21-AI.

In a modified version of this experiment, the RAMP module that will be cloned will contain only some of the cas genes, where one or more of the genes are omitted.

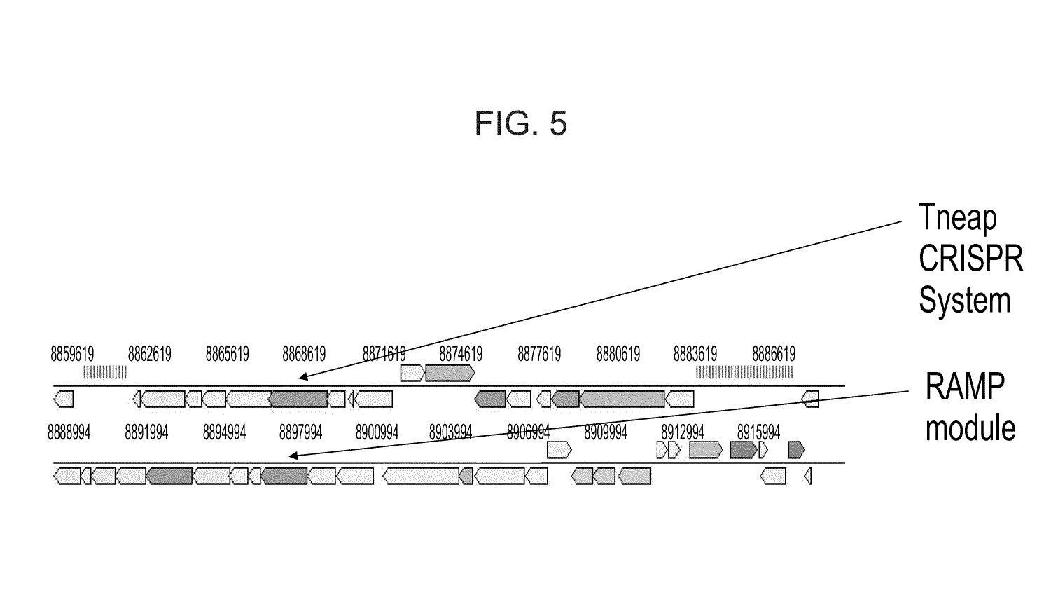

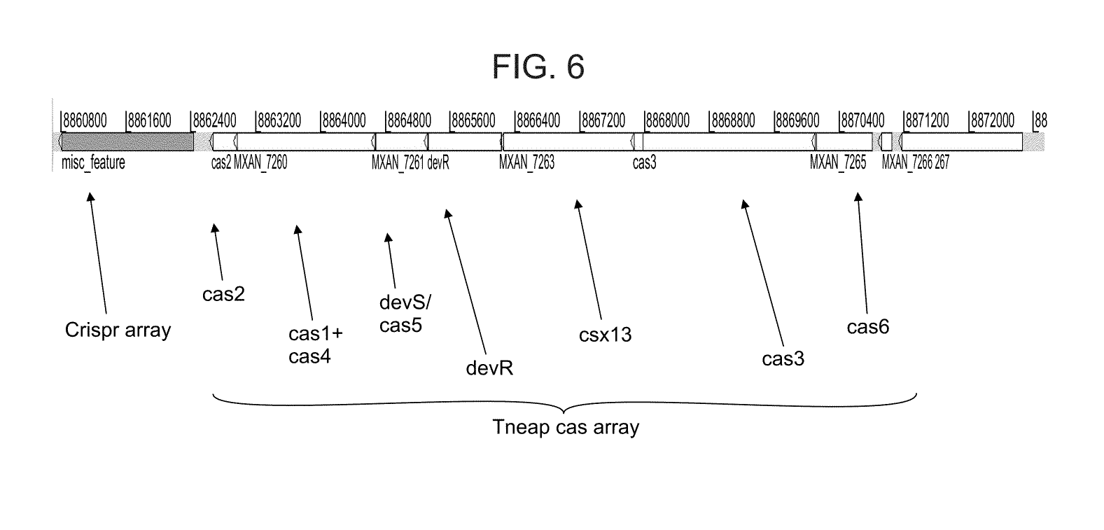

In a modified version of this experiment, another cas array will be cloned into the E. coli in conjugation with the plasmid described above. This cas array is of the Tneap subtype which resides on the genome of Myxococcus xanthus DK 1622 nearby the RAMP module (FIGS. 5-6), and might be involved in the processing of the repeat/spacer array of the RAMP module. SEQ ID NO: 1347 is the polynucleotide sequence of the cas array of the Myxococcus xanthus DK 1622 Genbank AC_{—}008095 RAMP module SEQ ID NO: 1348 provides the polynucleotide sequence of the cas array of the Myxococcus xanthus DK 1622 Genbank AC_{—}008095 CRISPR Tneap system.

The cas array of the Tneap subtype will be amplified from the genome of Myxococcus xanthus DK 1622 using the following primers

CGGATCCGTTGGCGCGGAGCGTCGGTTG (SEQ ID NO: 1352);

AAGCTTTCACAGCACCTTGAA (SEQ ID NO: 1353).

In a further modification, the Tneap cas array that will be cloned will contain only some of the cas genes, where one or more of the genes are omitted (for example, the Tneap cas array without cas 1 and cas2). SEQ ID No: 1349 provides the polynucleotide sequence of the cas array of the Myxococcus xanthus DK 1622 Genbank AC_{—}008095 CRISPR Tneap system without the cas1 and cas2 genes

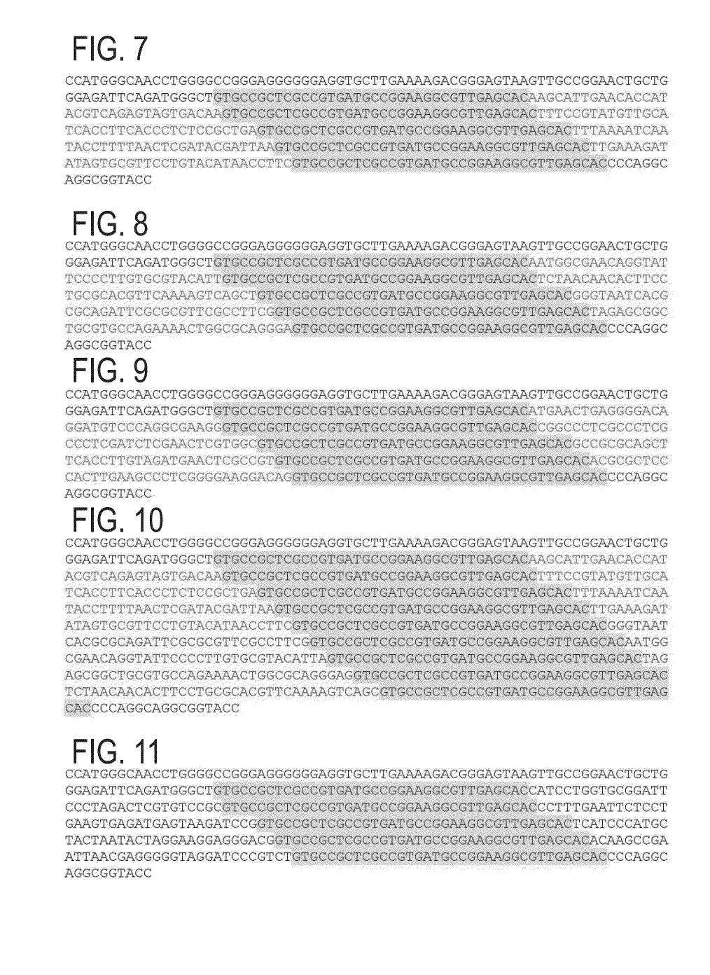

To allow silencing of the selected genes, DNA constructs containing CRISPR arrays will be cloned in another plasmid under the control of an inducible promoter and will also be transformed to the E. coli strain BL21-AI (FIGS. 7-10). Each of the CRISPR array DNA construct will contain one or more spacers directed against the antisense strand of the gene to be silenced. In the current example, the number of targeting spacers per gene is 4.

To show that more than one gene can be silenced by the same RAMP module in parallel, a CRISPR array will be designed that targets both GFP and malF at the same time (FIG. 10).

As a control for the above described experiments a CRISPR array construct that does not target any E coli gene will also be transformed (FIG. 11).

Degradation of the RNA of the selected genes can be further verified by Northern Blot or quantitative PCR.

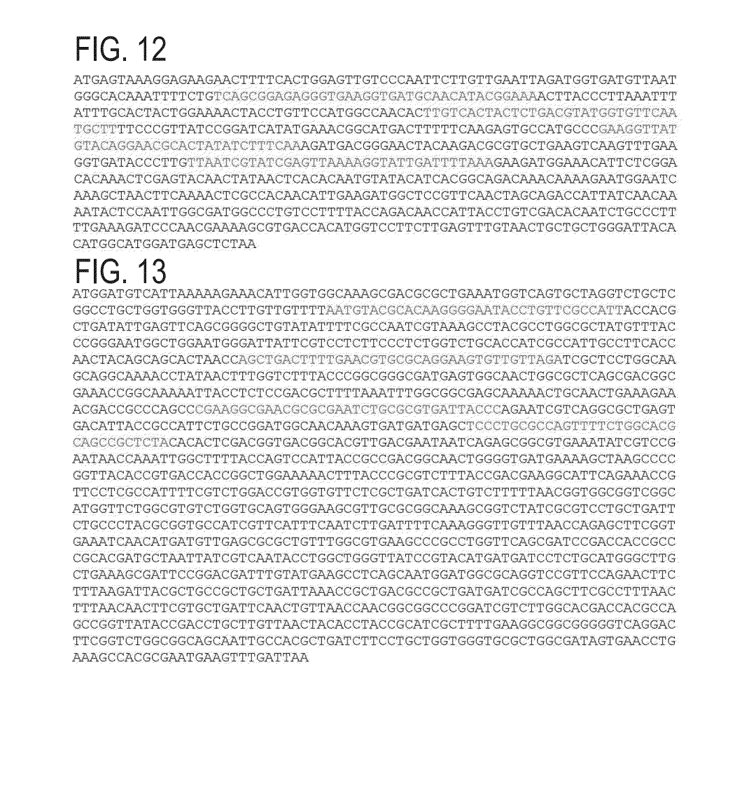

The CRISPR constructs for silencing GFP, RFP and malF expression in E. coli are illustrated in FIGS. 7-10. The GFP and malF sequences are illustrated in FIGS. 12-13.

### Example 3

### Down-Regulation of Genes in E. coli Bacteria Using a Neisseria sicca Derived CRISPR Polynucleotide Sequence

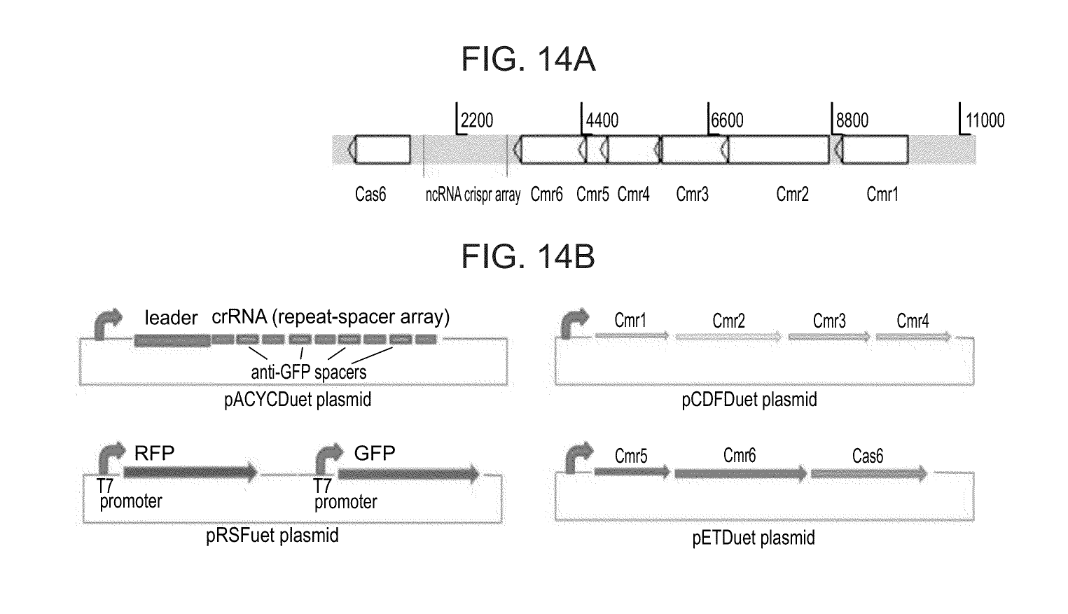

The RAMP CRISPR module of Neisseria sicca ATCC29256, a mesophilic bacteria isolated from the pharyngeal mucosa of healthy man was selected for experimentation. A draft genome of this organism is available, and in this genome (NCBI locus NZ_ACK002000045) a RAMP module was identified. This module was cloned into E. coli BL21 (DE3) on the pET-Duet compatible plasmids system, so that the CRISPR array (crRNA) was on one plasmid, and the cas genes were divided to two operons on two plasmids, all under an expression control inducible by IPTG (FIG. 14). Since the GC content and codon bias in the genomes of origin of each of these systems differ than that of E. coli, each system was synthesized with GC-content and codon-optimization for optimal expression in E. coli.

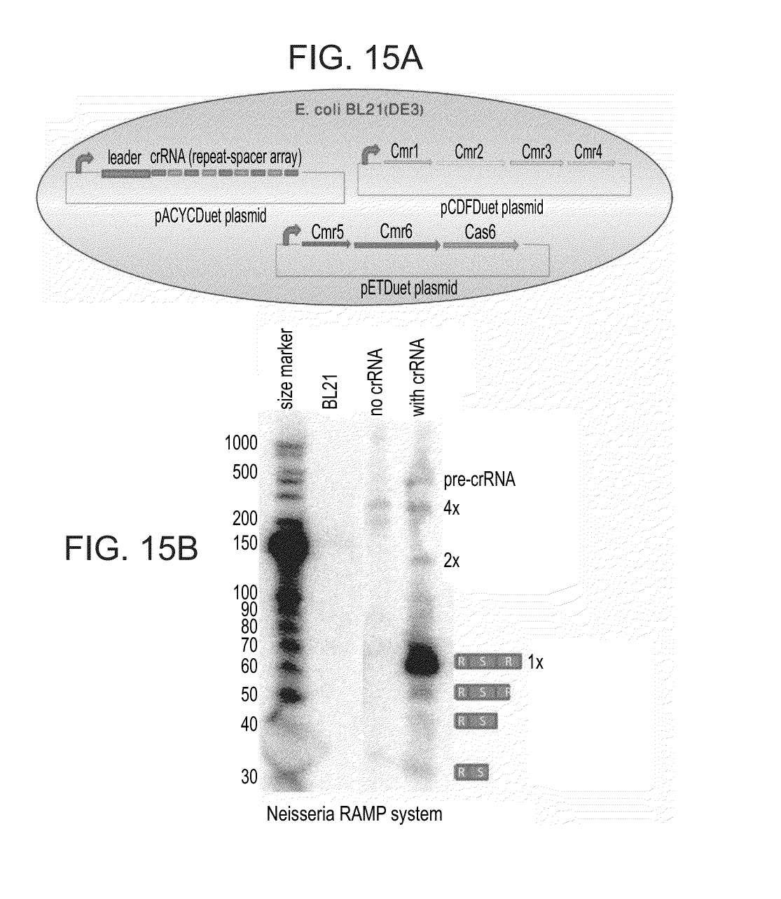

To test whether the RAMP system cloned into E. coli was active, the expression of each system was induced in E. coli BL21 using 0.1 mM IPTG. Total RNA was extracted and Northern blots were performed with probes designed against one of the spacers in the array. From the Northern blots, clear processing of the pre-crRNA was observed, confirming that the protein responsible for pre-crRNA processing are active (FIG. 15B). Moreover, similar to the Pyrococcus furiosus RAMP, two shorter RNA products were observed, probably corresponding to further trimming of the 3′ end of the crRNA (FIG. 15B). Since this further trimming is performed by one or more proteins in the Cmr complex, this confirms that this systems are active within E. coli, at least at the level of crRNA processing.

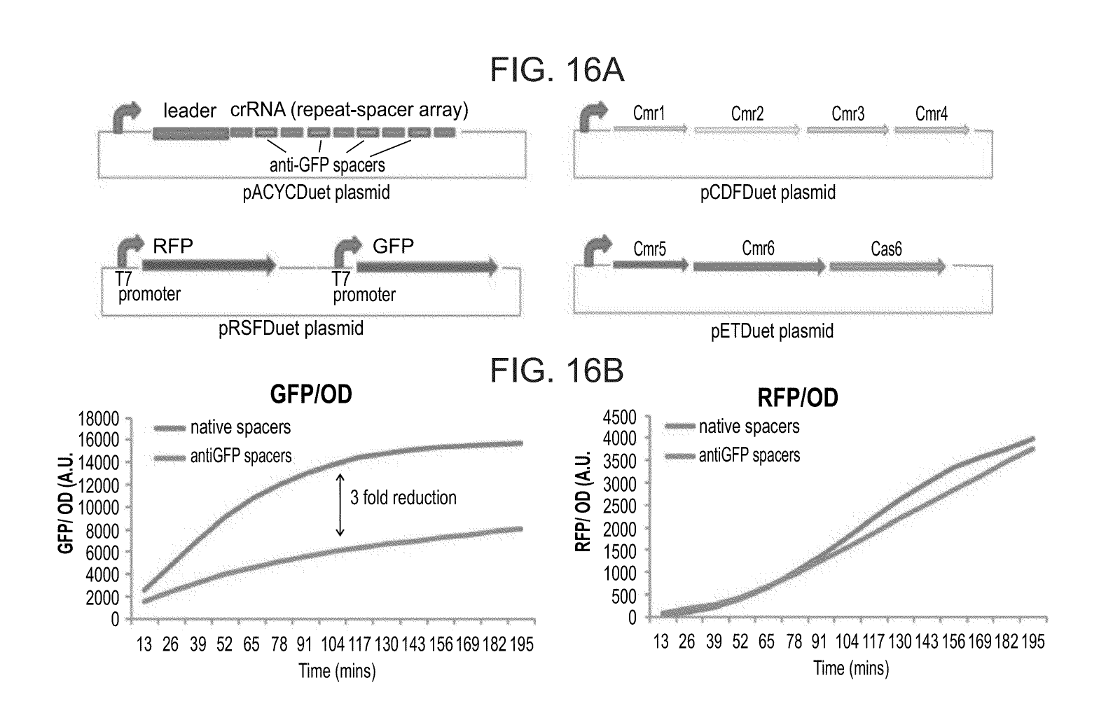

Next, the present inventors designed an experimental system that would allow high throughput measurements of RNA silencing. For this, they first engineered into the CRISPR array four spacers that target green fluorescence protein (GFP). Next, they prepared a two-gene construct, which includes GFP and RFP, cloned on a pRSF-Duet plasmid that is compatible with the plasmids carrying the CRISPR system (FIG. 16A). This system allows fluorescence-based measurements of GFP and RFP expression following CRISPR activation. If RNA-silencing is active, reduction in GFP expression (but not in RFP expression) will be observed. The experiments were performed in a 96-well plate format, where both O.D. and fluorescence are continuously measured at 37° C. by a robotic plate reader (Tecan Infinite 200 Pro), allowing the testing of up to 96 RAMP variants in less than one day.

Fluorescence levels of GFP and RFP following induction of the RAMP module that expresses crRNA with four spacers targeting different regions in the GFP gene were tested (FIG. 16A). As a control, a similar measurement was performed except that the crRNA expresses four native spacers (not matching any gene in the E. coli genome). A clear reduction of GFP levels was measured, but only a subtle reduction in RFP, suggesting RNA-targeting of the GFP (FIG. 16B). These results provide support for the hypothesis that RAMPs can be used for RNA-silencing in bacteria.

In order to make a RAMP-based system in which any gene of choice could be targeted, the present inventors designed a unique repeat-spacer array construct, based on the repeat sequences of the Neisseria sicca RAMP module, in which any spacer of choice could be inserted using a simple blunt-end ligation and cloning reaction (FIG. 17). This construct comprises:
1. A leader sequence upstream of the first repeat;
2. Four repeats and two spacers originating from Neisseria sicca (not targeting genes in E. coli);
3. Two consecutive repeats that naturally comprise of a unique blunt-end restriction site (for the ZraI restriction enzyme) which once cleaved can allow the insertion of any spacer to the CRISPR array using blunt end ligation;
4. A targeting-spacer, non-native to the original Neisseria array that can complement any selected target gene. This component can be inserted to the original array via ligation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An isolated polynucleotide, comprising
(i) a clustered, regularly interspaced short palindromic repeat (CRISPR) array nucleic acid sequence wherein at least one spacer of said CRISPR is sufficiently complementary to a portion of at least one prokaryotic gene so as to down-regulate expression of said prokaryotic gene; and
(ii) a nucleic acid sequence encoding at least one CRISPR associated (CAS) polypeptide of a repeat associated mysterious protein (RAMP) family.

2. The isolated polynucleotide of claim 1, wherein said at least one spacer comprises at least two spacers, each being sufficiently complementary to a portion of different prokaryotic genes so as to down-regulate expression of said different prokaryotic genes.

3. The isolated polynucleotide of claim 1, wherein said at least one spacer comprises 26-72 base pairs.

4. The isolated polynucleotide of claim 1, wherein said prokaryotic gene is a bacterial gene.

5-13. (canceled)

14. The isolated polynucleotide of claim 1, further comprising a nucleic acid sequence encoding a CRISPR leader sequence.

15. The isolated polynucleotide of claim 1, wherein said at least one CRISPR associated (CAS) polypeptide of a RAMP family is of a mesophilic organism.

16-17. (canceled)

18. The isolated polynucleotide of claim 1, wherein said nucleic acid sequence encoding at least one CRISPR associated (CAS) polypeptide of a RAMP family comprises a sequence encoding a RAMP module.

19. (canceled)

20. A nucleic acid construct comprising the isolated polynucleotide of claim 1.

21. The nucleic acid construct of claim 20, comprising a nucleic acid sequence selected from the group consisting of SEQ ID NOs: 1, 6 and 11.

22. The nucleic acid construct of claim 20, comprising a nucleic acid sequence encoding at least one polypeptide having at sequence selected from the group consisting of SEQ ID NOs: 1354-1360.

23-26. (canceled)

27. A nucleic acid construct system comprising:
(i) a first nucleic acid construct comprising an isolated polynucleotide having a clustered, regularly interspaced short palindromic repeat (CRISPR) array nucleic acid sequence wherein at least one spacer of said CRISPR is sufficiently complementary to a portion of at least one prokaryotic gene so as to down-regulate expression of said prokaryotic gene; and
(ii) a second nucleic acid construct comprising an isolated polynucleotide having a nucleic acid sequence encoding at least one CRISPR associated (CAS) polypeptide of a repeat associated mysterious protein (RAMP) family.

28. The nucleic acid construct system of claim 27, wherein said at least one CAS polypeptide comprises an amino acid sequence selected from the group consisting of SEQ ID NOs: 1354-1360.

29. The nucleic acid construct system of claim 28, wherein said first nucleic acid construct comprises a leader sequence upstream to said at least one spacer as set forth in SEQ ID NO: 1374.

30. The nucleic acid construct system of claim 27, wherein a repeat sequence of said CRISPR array is as set forth in SEQ ID NO: 1369, 1373, 1374 or 1375.

31-34. (canceled)

35. A method of down-regulating expression of a gene of a prokaryotic cell, the method comprising introducing into the cell a CRISPR system polynucleotide encoding a CRISPR array and at least one CRISPR associated (CAS) polypeptide of a repeat associated mysterious protein (RAMP) family, wherein a spacer of said CRISPR array is sufficiently complementary with a portion of the gene to down-regulate expression of the gene, thereby down-regulating expression of gene of a prokaryotic cell.

36. The method of claim 35, wherein the gene is not introduced into the cell by a bacteriophage.

37. The method of claim 35, wherein the gene is integrated into a chromosome of the cell.

38. The method of claim 35, wherein the gene is endogenous to the prokaryotic cell.

39. The method of claim 35, wherein the gene is epichromosomal.

40. The method of claim 35, further comprising introducing into the cell a naïve CRISPR array system.

41-44. (canceled)
